(19) United States
(12) Patent Application Publication
(10) Pub. No.: US 2025/0078984 A1
(43) Pub. Date: Mar. 6, 2025

(54) INFORMATION PROCESSING APPARATUS, INFORMATION PROCESSING METHOD, AND NON-TRANSITORY RECORDING MEDIUM

(71) Applicants: Takayuki YAMAGUCHI, Osaka (JP); Yuuki OKA, Osaka (JP); Yoshihiko MIKI, Kyoto (JP); Makoto WATANABE, Hyogo (JP); Shu TANAKA, Osaka (JP)

(72) Inventors: Takayuki YAMAGUCHI, Osaka (JP); Yuuki OKA, Osaka (JP); Yoshihiko MIKI, Kyoto (JP); Makoto WATANABE, Hyogo (JP); Shu TANAKA, Osaka (JP)

(21) Appl. No.: 18/803,941

(22) Filed: Aug. 14, 2024

(30) Foreign Application Priority Data
Sep. 1, 2023 (JP) 2023-142236

(51) Int. Cl. G16H 20/70; G16H 40/20

(52) U.S. Cl. CPC G16H 20/70; G16H 40/20

(57) ABSTRACT

An information processing apparatus includes circuitry to acquire information including biological information of a user, and cause a display to display at least one of a subjective indicator of total mental fatigue, a subjective indicator of concentration, or multiple types of mental fatigue determined based on the acquired information and a learned model. The learned model has learned relations between biological information acquired in advance and the subjective indicator of total mental fatigue, the subjective indicator of concentration, or each of the multiple types of mental fatigue.

### CROSS-REFERENCE TO RELATED APPLICATIONS

This patent application is based on and claims priority pursuant to 35 U.S.C. § 119 (a) to Japanese Patent Application No. 2023-142236, filed on Sep. 1, 2023, in the Japan Patent Office, the entire disclosure of which is hereby incorporated by reference herein.

### BACKGROUND

### Technical Field

The present disclosure relates to an information processing apparatus, an information processing method, and a non-transitory recording medium.

### Related Art

A mental and physical condition estimation system is disclosed. The mental and physical condition estimation system includes a heart rate information acquisition unit for acquiring heart rate information that is information related to a heart rate of a human subject; a heart rate variability calculation unit for calculating heart rate variability of a very low frequency (VLF) component from the acquired heart rate information; and a mental and physical condition estimation unit for estimating a level of concentration and effort of the human subject from a calculated value of the heart rate variability.

### SUMMARY

In one embodiment, an information processing apparatus includes circuitry to acquire information including biological information of a user; and cause a display to display at least one of a subjective indicator of total mental fatigue, a subjective indicator of concentration, or multiple types of mental fatigue determined based on the acquired information and a learned model. The learned model has learned relations between biological information acquired in advance and the subjective indicator of total mental fatigue, the subjective indicator of concentration, or each of the multiple types of mental fatigue.

In another embodiment, an information processing method includes acquiring information including biological information of a user; and causing a display to display at least one of a subjective indicator of total mental fatigue, a subjective indicator of concentration, or multiple types of mental fatigue determined based on the acquired information and a learned model. The learned model has learned relations between biological information acquired in advance and the subjective indicator of total mental fatigue, the subjective indicator of concentration, or each of the multiple types of mental fatigue.

In another embodiment, a non-transitory recording medium stores a plurality of instructions which, when executed by one or more processors, causes the processors to perform a method. The method includes acquiring information including biological information of a user; and causing a display to display at least one of a subjective indicator of total mental fatigue, a subjective indicator of concentration, or multiple types of mental fatigue determined based on the acquired information and a learned model. The learned model has learned relations between biological information acquired in advance and the subjective indicator of total mental fatigue, the subjective indicator of concentration, or each of the multiple types of mental fatigue.

### BRIEF DESCRIPTION OF THE DRAWINGS

A more complete appreciation of embodiments of the present disclosure and many of the attendant advantages and features thereof can be readily obtained and understood from the following detailed description with reference to the accompanying drawings, wherein:

FIG. 1 is a diagram illustrating an overall configuration of an information processing system according to one embodiment;

FIG. 2 is a diagram illustrating a hardware configuration of an information analysis device and a terminal device according to one embodiment;

FIG. 3 is a diagram illustrating a hardware configuration of a detector according to one embodiment;

FIG. 4 is a diagram illustrating a functional configuration of the information processing system according to one embodiment;

FIG. 5A is a sequence diagram illustrating a process according to a first embodiment;

FIG. 5B is a sequence diagram illustrating a process according to the first embodiment;

FIG. 6 is an illustration of a screen displayed on a display of the terminal device according to one embodiment;

FIG. 7 is a graph illustrating a respiratory waveform according to one embodiment;

FIG. 8 is table illustrating datasets for training, which are used for estimation, according to one embodiment;

FIG. 9 is a table illustrating relationship between explanatory variables, intermediate characteristics, and objective variables according to one embodiment;

FIG. 10 is a graph illustrating relationship between fatigue types and respiratory information according to one embodiment;

FIG. 11 is a chart illustrating a seating determination method according to one embodiment;

FIG. 12 is a chart illustrating a conversation determination method according to one embodiment;

FIG. 13 is a graph illustrating a visualization of concentration levels over time according to one embodiment;

FIGS. 14A and 14B are each a graph illustrating a visualization of fatigue levels on a weekly basis according to one embodiment;

FIG. 15 is a graph illustrating a visualization of fatigue levels as remaining energy levels according to one embodiment;

FIG. 16 is a graph illustrating a visualization of fatigue levels and concentration levels on a weekly basis according to one embodiment;

FIG. 17 is a sequence diagram illustrating a process according to a second embodiment;

FIG. 18 is an illustration of a schedule table on which concentration levels are superimposed, according to one embodiment;

FIG. 19 is a sequence diagram illustrating a process according to a third embodiment;

FIG. 20 is a flowchart illustrating a process according to the third embodiment;

FIG. 21 is a graph illustrating changes in concentration level for tasks according to one embodiment;

FIG. 22 is a table illustrating tasks and concentration time constants according to one embodiment;

FIG. 23 is a table illustrating fatigue types and recovery actions associated with the fatigue types according to one embodiment; and

FIG. 24 is an illustration of a display screen on the terminal device, presenting a recovery action, according to one embodiment.

The accompanying drawings are intended to depict embodiments of the present disclosure and should not be interpreted to limit the scope thereof. The accompanying drawings are not to be considered as drawn to scale unless explicitly noted. Also, identical or similar reference numerals designate identical or similar components throughout the several views.

### DETAILED DESCRIPTION

In describing embodiments illustrated in the drawings, specific terminology is employed for the sake of clarity. However, the disclosure of this specification is not intended to be limited to the specific terminology so selected and it is to be understood that each specific element includes all technical equivalents that have a similar function, operate in a similar manner, and achieve a similar result.

Referring now to the drawings, embodiments of the present disclosure are described below. As used herein, the singular forms “a,” “an,” and “the” are intended to include the plural forms as well, unless the context clearly indicates otherwise.

Embodiments of the present disclosure will be described with reference to the drawings. In the description of the drawings, the same elements are denoted by the same reference numerals, and the redundant description thereof will be omitted.

### First Embodiment

### Overall Configuration of Information Processing System

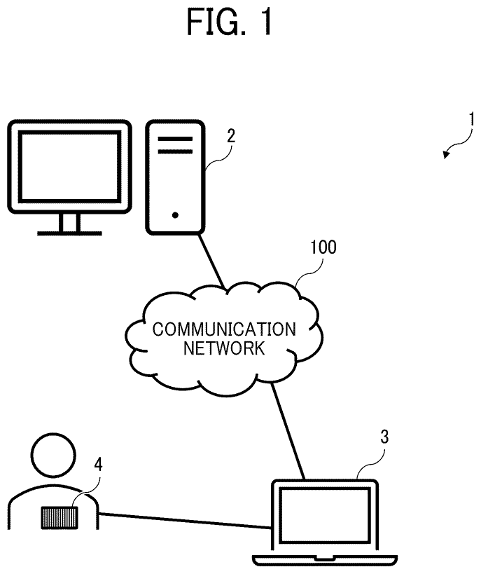

FIG. 1 is a diagram illustrating an example of an overall configuration of an information processing system 1. As illustrated in FIG. 1, the information processing system 1 includes devices and terminals such as an information analysis device 2, a terminal device 3 (e.g., a communication terminal), and a detector 4.

A communication network 100 is a communication network through which an unspecified number of types of communication are performed, and includes the Internet, an intranet, and a local area network (LAN). The communication network 100 may include a wired communication network and a wireless communication network. The wireless communication network may be based on a wireless communication standard such as fourth generation (4G), fifth generation (5G), Worldwide Interoperability for Microwave Access (WiMAX), or Long Term Evolution (LTE).

The information analysis device 2 performs information analysis and serves as a storage device in the information processing system 1. The information analysis device 2 is a so-called server. The information analysis device 2 has an estimation function of estimating, for example, the fatigue state or the concentration state of a user, and a schedule optimization computation function of computing a schedule optimal for the user. The information analysis device 2 further has an action recommendation computation function of computing an action recommend for the user and prompting the user to perform the recommend action. The results of computation are accumulated in a storage unit. The information analysis device 2 may be a general-purpose personal computer (PC) or a mobile laptop PC.

The terminal device 3 is a communication terminal to be used by a user of the information processing system 1. The terminal device 3 receives (acquires) and inputs information on the user to be used by the information processing system 1. The information input includes input through a keyboard of a computer terminal and signals from a sensor worn by the user. The terminal device 3 also provides information to the user. The terminal device 3 includes a visualization unit that outputs the result of a computation unit or information accumulated in a storage unit, and displays a computation result or the accumulated information to the user on the user's terminal. The terminal device 3 is implemented by an information processing apparatus (computer system) configured to perform communication and including typical computer components such as an operating system (OS) and is a part of the information processing system 1.

The terminal device 3 may be a general-purpose PC, a mobile laptop PC, a mobile phone, a smartphone, a tablet terminal, or a communication terminal. Alternatively, the terminal device 3 is a communication device or a communication terminal on which browser software and various types of software, such as applications, operate.

The detector 4 acquires biological information of the user and continuously senses biological information of the user during work. One example of the detector 4 is a contact respiration sensor for measuring a respiratory signal of the user. The respiration sensor has a wireless communication function such as a Bluetooth® enabled function to wirelessly communicate with a smartphone or a PC that the user uses during work. The respiration sensor as the detector 4 constantly detects a respiratory signal of the user and transmits a detected respiratory waveform to the smartphone or the PC via wireless communication.

### Hardware Configuration

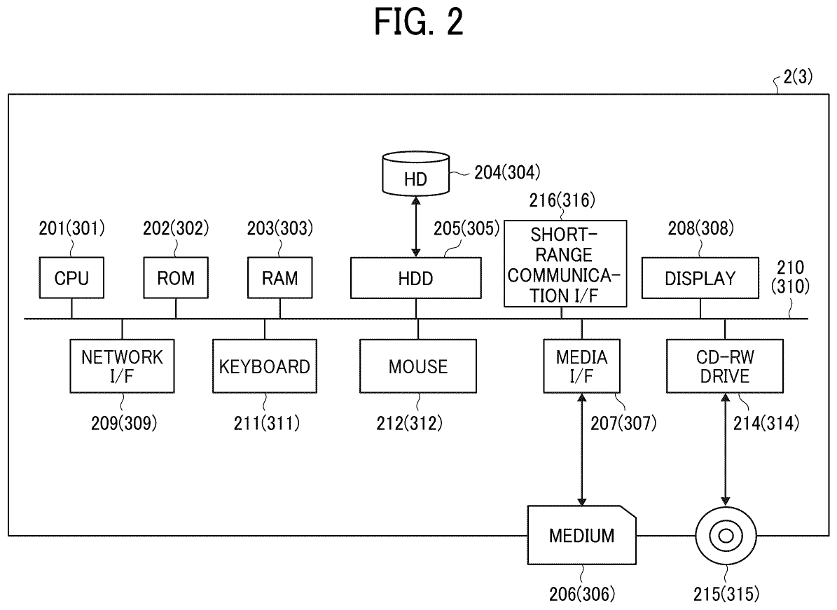

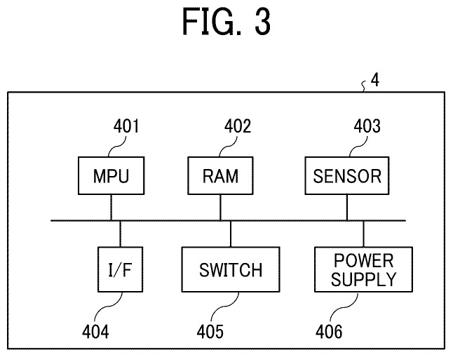

The hardware configuration of communication terminals or devices included in the information processing system 1 according to an embodiment will be described with reference to FIGS. 2 and 3. Of the hardware elements of a communication terminal or a device illustrated in FIGS. 2 and 3, an element may be added or deleted as appropriate.

### Hardware Configuration of Information Analysis Device 2 and Terminal Device 3

FIG. 2 is a diagram illustrating an example hardware configuration of the information analysis device 2 and the terminal device 3. Since the information analysis device 2 and the terminal device 3 have substantially the same configuration, the configuration of the terminal device 3 will mainly be described below, with the components of the information analysis device 2 designated by reference numerals in parentheses. As illustrated in FIG. 2, the information analysis device 2 and the terminal device 3 are each implemented by, for example, a computer. The terminal device 3 (the information analysis device 2) includes a central processing unit (CPU) 301 (201), a read-only memory (ROM) 302 (202), a random-access memory (RAM) 303 (203), a display 308 (208), and a network interface (I/F) 309 (209). The terminal device 3 (the information analysis device 2) further includes a short-range communication I/F 316 (216), a medium 306 (206), a media I/F 307 (207), a keyboard 311 (211), a mouse 312 (212), a hard disk (HD) 304 (204), a hard disk drive (HDD) controller 305 (205), and a bus line 310 (210).

The CPU 301 (201) controls the overall operation of the terminal device 3 (the information analysis device 2). The ROM 302 (202) stores, for example, a program used for driving the CPU 301 (201). The RAM 303 (203) is used as a work area for the CPU 301 (201). The display 308 (208) displays various types of information such as a cursor, a menu, a window, characters, or an image. In the present embodiment, the display 308 (208) is an example of a display means. The short-range communication I/F 316 (216) is a communication circuit for performing data communication with a communication apparatus or a communication terminal including a wireless communication interface such as a near-field communication (NFC) interface, a Bluetooth® interface, or a Wi-Fi® interface.

The HD 304 (204) stores various types of data such as programs. The HDD controller 305 (205) controls the reading or writing of various types of data from or to the HD 304 (204) under the control of the CPU 301 (201). The terminal device 3 (the information analysis device 2) may have a hardware configuration including a solid state drive (SSD), a compact disc rewritable (CD-RW) drive 314 (214), and a compact disc (CD) 315 (215) in place of the HD 304 (204) and the HDD controller 305 (205).

The network I/F 309 (209) is an interface for performing data communication using the communication network 100. The keyboard 311 (211) and the mouse 312 (212) are types of input units used by the user to perform an operation such as pressing, clicking, or tapping a predetermined button or icon arranged on the display 308 (208) to operate the terminal device 3 (the information analysis device 2). The media I/F 307 (207) reads or writes (stores) data from or to the medium 306 (206) such as a flash memory. The bus line 310 (210) is, for example, an address bus or a data bus for electrically connecting the components such as the CPU 301 (201) to each other.

The programs described above may be distributed as files in an installable or executable format recorded on a computer-readable recording medium or downloaded via a network. Examples of the recording medium include a compact disc recordable (CD-R), a digital versatile disc (DVD), a Blu-ray Disc®, a secure digital (SD) card, and a universal serial bus (USB) memory. The recording medium may be provided in the form of a program product to domestic or foreign users.

For example, the information analysis device 2 implements an information analysis method according to one embodiment in response to the execution of a program according to one embodiment.

### Hardware Configuration of Detector 4

FIG. 3 is a diagram illustrating an example hardware configuration of the detector 4. As illustrated in FIG. 3, the detector 4 is implemented by, for example, a microcomputer. The detector 4 includes a micro processing unit (MPU) 401, a RAM 402, an I/F 404, a sensor 403, a switch 405, and a power supply 406.

The sensor 403 includes, for example, an inertial measurement sensor unit, a microphone, and a temperature detection sensor. The inertial measurement sensor unit is a three-dimensional inertial motion capture sensor. Specifically, the inertial measurement sensor unit captures translational motion and rotational motion in triaxial orthogonal directions. An acceleration sensor and a gyro sensor are used. A geomagnetic sensor is also used to indicate the sensor position and direction as a Euler angle.

The microphone is implemented by a micro-electro-mechanical systems (MEMS) microphone or an electret condenser microphone. In one embodiment, the microphone acquires audio information. The audio information includes information indicating whether the user has a conversation, respiratory sounds of the user, and environmental sounds around the user.

The MPU 401 includes a control unit that is implemented by, for example, an integrated circuit chip including a microcomputer, a logic device such as a field-programmable gate array (FPGA), or a combination of an integrated circuit chip and a logic device. The control unit performs, for example, processing of data obtained by the sensor 403 and issues instructions of data communication to the system. The switch 405 enables external operations such as turning on and off the power supply and resetting the power supply 406.

The I/F 404 is an interface through which the detector 4 communicates with the terminal device 3, and is configured to communicate data via wireless communication, such as Wi-Fi® or Bluetooth® connection, or via wired connection. When the user wearing the detector 4 is performing a wireless communication operation, the detector 4 may serve as a slave device for wireless communication on the user end, and a master device for wireless communication may be separately included in a wearable device such as a smartphone or a smart watch. In one embodiment, the detector 4 communicates with the terminal device 3 via the smartphone or the like.

The detector 4 acquires biological information of the user and is attached to the chest or waist of the user. The detector 4 is attached to any part of the body as long as body motion caused by breathing can be captured. In one example, the detector 4 is hooked with a clip to be secured to the user's clothing including a belt. In another example, the detector 4 is secured by a belt that is wound around the chest or abdomen. In another example, the detector 4 is coupled to a neck strap that hangs from the neck, and is secured.

### Configuration of Functional Blocks

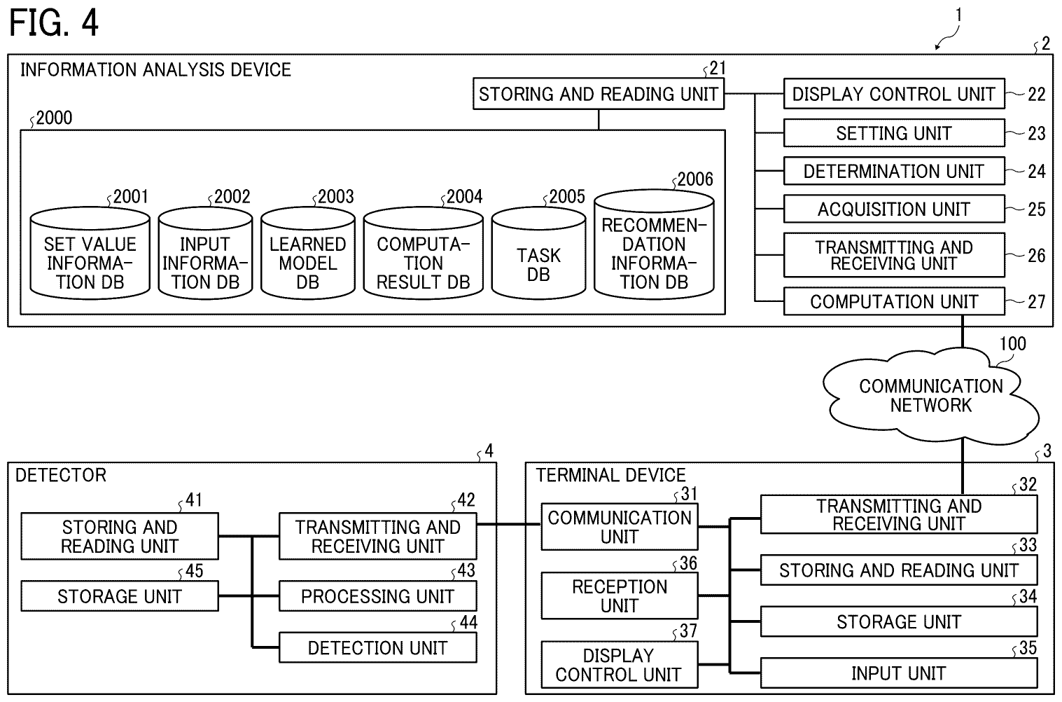

FIG. 4 is a functional block diagram of the system according to the present embodiment. The information processing system 1 includes the information analysis device 2, the terminal device 3, and the detector 4. The functional blocks of the information analysis device 2, the terminal device 3, and the detector 4 will be described in order.

### Functional Configuration of Information Analysis Device 2

First, the functional configuration of the information analysis device 2 will be described. As illustrated in FIG. 4, the information analysis device 2 includes a storing and reading unit 21, a display control unit 22, a setting unit 23, a determination unit 24, an acquisition unit 25, a transmitting and receiving unit 26, and a computation unit 27. Each of the functional units described above is implemented by one or more of the hardware resources illustrated in FIG. 2 that operate in response to an instruction from the CPU 201 according to a program for the information analysis device 2 loaded into the RAM 203 from at least one of the ROM 202, the HD 204, and the medium 206. The information analysis device 2 further includes a storage unit 2000. The storage unit 2000 is implemented by at least one of the ROM 202, the HD 204, and the medium 206 illustrated in FIG. 2. For example, a communication program (communication application) for performing communication with the terminal device 3 via the communication network 100 and a browser application are installed and stored in the storage unit 2000.

Then, the functional units of the information analysis device 2 will be described in detail. The transmitting and receiving unit 26 of the information analysis device 2 illustrated in FIG. 4 is implemented by, for example, processing performed by the CPU 201 on the network I/F 209 and the short-range communication I/F 216. The transmitting and receiving unit 26 transmits and receives various types of data (or information) to and from the terminal device 3 via the communication network 100. The display control unit 22 is implemented by, for example, processing of the CPU 201 on the display 208 of the information analysis device 2, and performs display control of various screens and information (data) on the information analysis device 2. The display control unit 22 displays a display screen generated by, for example, HyperText Markup Language (HTML) on the display 208 of the information analysis device 2 using, for example, a browser. In the present embodiment, the display control unit 22 is an example of a display control means, and the display 208 is an examples of display.

The setting unit 23 is implemented by, for example, processing performed by the CPU 201. The setting unit 23 performs a process to set setting information input by the user. In the present embodiment, the setting unit 23 is an example of a setting means.

The determination unit 24 is implemented by, for example, processing performed by the CPU 201. The determination unit 24 performs various determination processes of the information analysis device 2. In the present embodiment, the determination unit 24 is an example of a determination means.

The acquisition unit 25 is implemented by, for example, processing performed by the CPU 201. The acquisition unit 25 acquires, for example, biological information from the detector 4. The acquisition unit 25 also acquires user information input via, for example, the keyboard 311 and the mouse 312 of the terminal device 3. In the present embodiment, the acquisition unit 25 is an example of an acquisition means.

The storing and reading unit 21 is implemented by, for example, processing performed by the CPU 201 on at least one of the ROM 202, the HD 204, and the medium 206. The storing and reading unit 21 stores various types of data (or information) in the storage unit 2000 and reads various types of data (or information) from the storage unit 2000. In the present embodiment, the storing and reading unit 21 is an example of a storing and reading means.

Next, databases included in the information analysis device 2 (information processing apparatus) will be described. A set value information database (DB) 2001 accumulates, for example, threshold information set in advance, used by an estimation unit of the computation unit 27.

An input information DB 2002 accumulates user information obtained by an input device 35 of the terminal device 3 or biological information obtained by a biological information acquisition means. The input information DB 2002 also includes information input by the user using the keyboard 311 of the terminal device 3, information selected by the user operating the mouse 312 from information displayed on the display 308, and information acquired from another application used by the user by using an application programming interface (API).

The user information in this disclosure is, for example, context information, task information, or attribute information of the user.

The computation unit 27 has the estimation function of estimating, for example, the fatigue state or the concentration state of a user, the schedule optimization computation function of computing a schedule optimal for the user, and the action recommendation computation function of computing an action recommend for the user and prompting the user to perform the recommend action. A computation result DB 2004 accumulates a schedule optimization computation result obtained by the computation unit 27, and an estimation result of, for example, the fatigue state or concentration state of the user.

A learned model DB 2003 includes training datasets and a learning algorithm. The training datasets represent the relationship between biological information to be learned and corresponding fatigue types, fatigue levels, and concentration levels.

A task DB 2005 stores a time constant of a fatigue level unique to each task. The time constant of the fatigue level is a quantitative indicator of the rate of increase in fatigue level, which differs depending on the task. In this embodiment, the task DB 2005 stores in a table various work items such as meetings, investigation tasks, programming tasks, and paper writing tasks, and time constants thereof.

A recommendation information DB 2006 accumulates information used to recommend a concentration action or a recovery action for a computation result of a fatigue type or a fatigue state. The information includes, for each of five fatigue types, a suitable recovery action. The information further includes a plurality of types of recovery actions such that a recovery action is associated with each degree of fatigue in each fatigue type.

In the information processing system 1 described above, the computation unit 27 and the storage unit 2000 may reside on a cloud server. The cloud server is a server that provides cloud computing resources.

### Functional Configuration of Terminal Device 3

Next, the functional configuration of the terminal device 3 will be described. As illustrated in FIG. 4, the terminal device 3 includes a communication unit 31, a transmitting and receiving unit 32, a storing and reading unit 33, a storage unit 34, an input unit 35, a reception unit 36, and a display control unit 37. Each of the functional units described above is implemented by one or more of the hardware resources illustrated in FIG. 2 that operate in response to an instruction from the CPU 301 according to a program for the terminal device 3 loaded into the RAM 303 from at least one of the ROM 302, the HDD controller 305, and the medium 306.

The terminal device 3 may be implemented by a single computer, such as a general-purpose PC or a mobile laptop PC, or a plurality of computers such that the components (functions or means) of the terminal device 3, such as a storage, are assigned to the plurality of computers as appropriate. In one embodiment, all or some of the functions of the terminal device 3 are implemented by a server computer residing on a cloud network or a server computer residing on an on-premises network. In another embodiment, the terminal device 3 is a communication device or a communication terminal on which software such as browser software operates.

The display control unit 37 is implemented by, for example, processing performed by the CPU 301 on the display 308 of the terminal device 3. The display control unit 37 controls the terminal device 3 to display various screens and information (or data). Further, the display control unit 37 causes the display 308 of the terminal device 3 to display a display screen generated in, for example, HTML by using, for example, a browser. In the present embodiment, the display control unit 37 is an example of a display control means.

### Functional Configuration of Detector 4

Next, the functional configuration of the detector 4 will be described. As illustrated in FIG. 4, the detector 4 includes a transmitting and receiving unit 42, a processing unit 43, a detection unit 44, a storing and reading unit 41, and a storage unit 45. Each of the functional units described above is implemented by one or more of the hardware resources illustrated in FIG. 3 that operate in response to an instruction from the MPU 401 according to a program for the detector 4 loaded into the RAM 402.

The detector 4 is a detection device that continuously senses biological information of the user during work. One example of the detector 4 is a contact respiration sensor for measuring a respiratory signal of the user.

As used herein, the term “respiratory signal” refers to a signal generated in response to a respiratory activity such as inhalation or exhalation. The movement of the lungs causes contraction of the chest or movement of respiratory muscles such as the diaphragm and intercostal muscles near the abdomen when the lungs take in oxygen from the air. Examples of the contact respiration sensor include a band-type respiration sensor to be worn on the chest or abdomen of the user.

Examples of the contact respiration sensor also include a mask-type respiration sensor that senses a change in temperature around the mouth due to respiration, as well as the movement of the chest and the abdomen. In one embodiment, a small measurement device such as an inertial measurement unit is used as a respiration sensor. This configuration allows the movement of the chest and the abdomen due to respiration to be captured with a small wearable device. In another embodiment, radio waves or the like is used to capture a respiratory activity in a non-contact manner. The I/F 404 of the detector 4 has a wireless communication function such as a Bluetooth® enabled function to wirelessly communicate with a smartphone or a PC that the user uses during work. The detector 4 detects a respiratory signal of the user and transmits information on a detected respiratory waveform or an extracted feature value to the smartphone or the terminal device 3 via wireless communication, as appropriate.

### Sequence of Processes

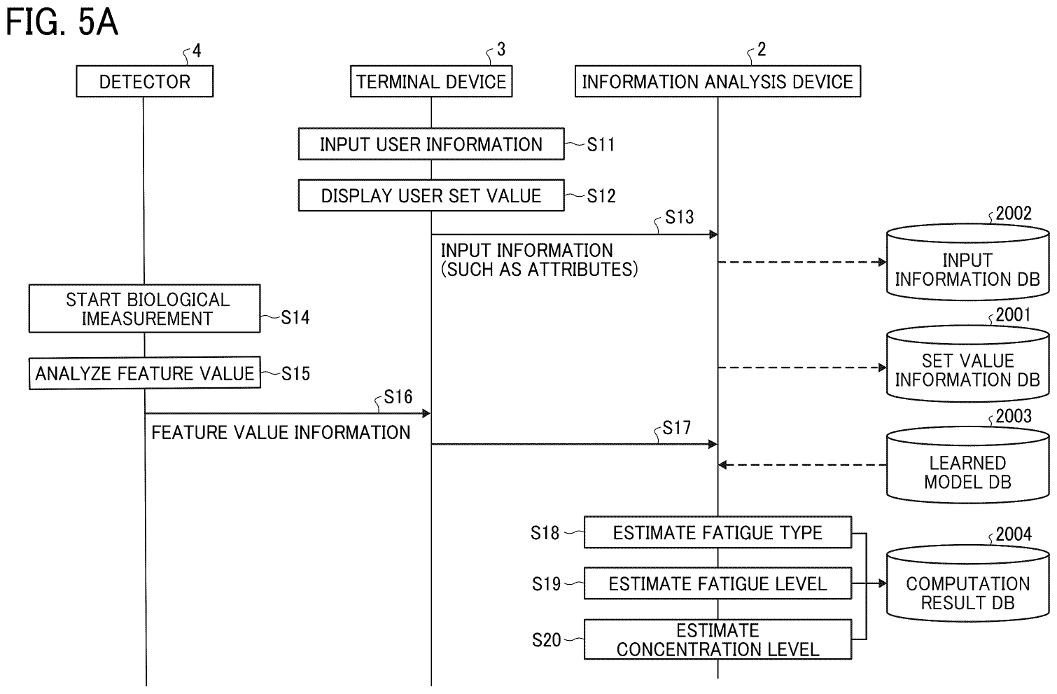

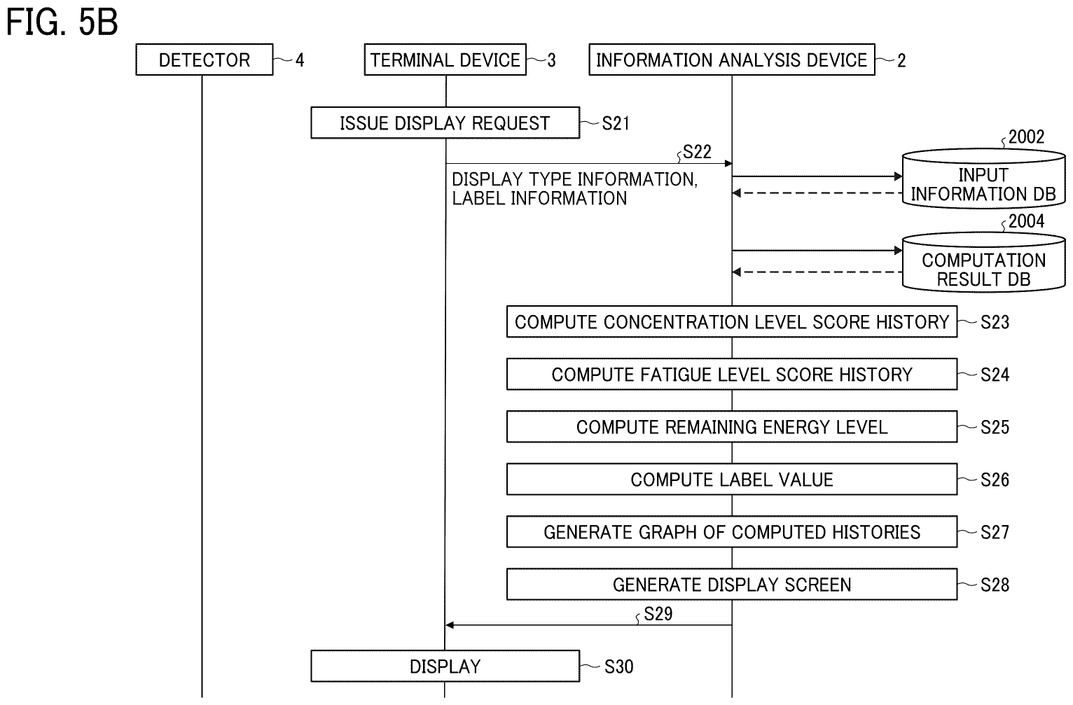

Next, the entire processing flow of the information processing system 1 according to the present embodiment will be described with reference to a sequence diagram illustrated in FIGS. 5A to 5B. The illustrated sequence of processes is performed by the information analysis device 2, the terminal device 3, and the detector 4. First, a user inputs user information to the terminal device 3 by using the keyboard 311 (step S11).

The user information in this step includes context information, task information, and attribute information of the user. The context information is information indicating a situation around the user. Examples of the context information include the current position of the user in the office, the current time, and the current work environment (e.g., temperature, humidity, atmospheric pressure, and illuminance). The task information is information included in a scheduler of the user.

Specifically, the task information includes the user's daily or weekly schedule, information on a task to be performed, and information on a task that has been performed. The information on a task that has been performed includes, for example, the achievement level of the task, the actual time taken for the task to complete, and the name of the actually performed task. The task information includes time information such as the user's scheduled work start time, scheduled work end time, actual work start time, and actual work end time. The attribute information is information such as gender and age. The user information further includes schedule information of another person related to the task in which the user is involved.

As a result, the input information is displayed as user set values (step S12).

The input user information is transmitted to the information analysis device 2 (step S13). The information analysis device 2 records the received user information in the input information DB 2002.

The detector 4 is attached to a part of the body of the user near the chest or the abdomen and starts measurement (step S14). Information detected by the detector 4 is processed by the processing unit 43 implemented by the MPU 401 of the detector 4. The detector 4 performs preprocessing such as noise removal, and extraction of a feature value described below (step S15). The setting information input by the user includes the timing or frequency with which the detector 4 transmits a feature value. The timing is measured by a timer in the MPU 401 of the detector 4. The extracted feature value is transmitted to the terminal device 3 via the transmitting and receiving unit 42 as appropriate (step S16). The feature value received by the terminal device 3 is transmitted to the information analysis device 2 as appropriate (step S17).

The information analysis device 2 receives the feature value transmitted from the detector 4. The information analysis device 2 estimates a fatigue type from the received feature value, the information obtained from the learned model DB 2003, and the user information obtained from the input information DB 2002 (step S18). Further, the information analysis device 2 estimates a fatigue level (step S19) and a concentration level (step S20), and records the obtained computation results in the computation result DB 2004.

A continuation of FIG. 5A is illustrated in FIG. 5B. When the user desires to display a measurement record, the user issues a display request from the terminal device 3 (step S21). The display request issued by the user includes display set values such as information on the type of display and the label to be displayed, and the information is transmitted to the information analysis device 2 (step S22).

The computation unit 27 of the information analysis device 2 computes the information in the computation result DB 2004 in response to the request from the user and generates a display screen. At this time, the information analysis device 2 computes a concentration level score history (step S23) and a fatigue level score history (step S24). Depending on the display request, the information analysis device 2 further computes the remaining energy level (step S25) or the label value (step S26). The information analysis device 2 performs a process of generating graphs of the computed histories (step S27). The computation unit 27 generates a display screen on which the generated graphs are laid out in an appropriate manner (step S28).

The transmitting and receiving unit 26 of the information analysis device 2 transmits the requested display screen to the terminal device 3 (step S29). The terminal device 3 displays the received display screen (step S30).

### Estimation Method

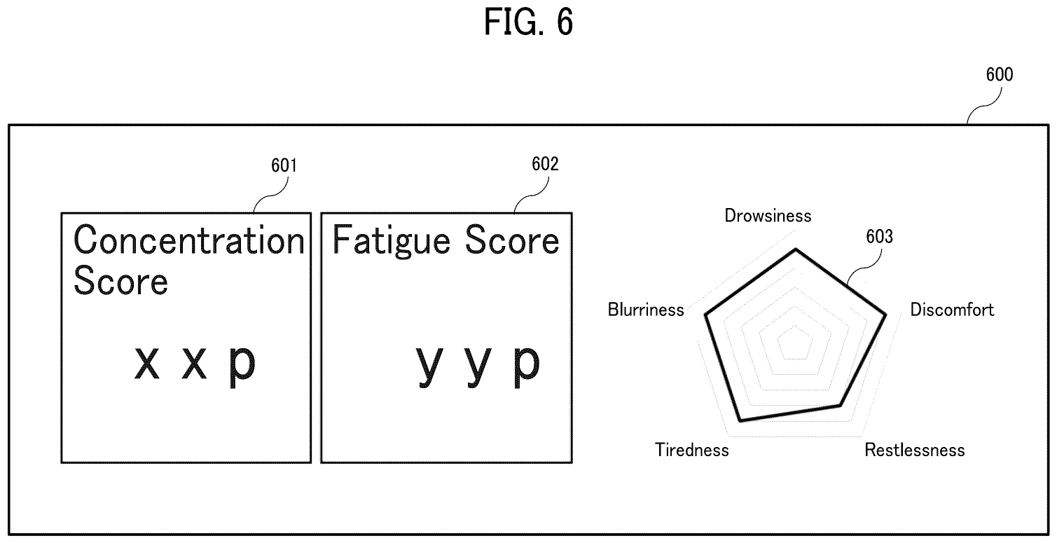

Next, a method for estimating a fatigue type, a fatigue level, and a concentration level will be described. FIG. 6 illustrates a display screen 600 as an example of visualized estimation results according to the present embodiment. The results include three components, namely, a concentration score 601, a fatigue score 602, and a radar chart 603 representing fatigue types. The concentration score 601 and the fatigue score 602 are relative and quantitative measures of how much the user is concentrating and how fatigued the user is, respectively, and indicate the levels of concentration and fatigue with respect to respective maximum values. Each of the maximum values is set to, for example, 100 or 10. The radar chart 603 represents five fatigue types: “drowsiness,” “discomfort,” “restlessness,” “tiredness,” and “blurriness.” Each type is represented in five stages and illustrated in a radar chart to facilitate a clear understanding of which item is relatively high. The items will be sequentially described in detail below.

### Representation of Fatigue Types

Next, a way in which the fatigue types are represented will be described. In one embodiment, a representation of the fatigue types is provided by the information analysis device 2. Alternatively, a representation of the fatigue types may be provided by the terminal device 3 or the detector 4. The fatigue type is estimated based on classification by using feature value information and a learned model included in the storage unit 2000 (i.e., the learned model DB 2003). In the present embodiment, five fatigue types, namely, “drowsiness,” “discomfort,” “restlessness,” “tiredness,” and “blurriness,” are used. Each of the five fatigue types indicates how the user subjectively feels for the corresponding fatigue type. The five fatigue types have their respective degrees. The current state of the user can be accurately represented by the respective degrees of the five fatigue types expressed in numeral as indicators.

According to the study by the inventors, categorizing fatigue into five types allows the user's condition to be reasonably represented using five fatigue indicators, and the user feels that these indicators are convincing and is highly satisfied, compared with evaluating fatigue with only one measure and indicating whether the level of fatigue is high or low. A high level of user satisfaction means there is a high consistency with the user's subjective evaluation. Conventional one or two indicators of fatigue severity have low consistency with subjective evaluation and fail to provide sufficient results. This is obtained from academic findings in psychology. In psychological studies, there are innumerable options such as how the five fatigue types are expressed, and whether the number of fatigue types to be used is five or four. Of such options, narrowing fatigue indicators to the five indicators makes the fatigue estimation more convincing. In the present embodiment, fatigue is categorized into five types to increase the consistency with subjective evaluation.

The five fatigue types are each evaluated using five scales, which will be described below. Specifically, the measure of drowsiness is evaluated with five items: heavy eyelids, preferring to be laying down, yawning, lack of motivation, and whole body weariness.

The measure of discomfort is evaluated with five items: headaches, heavy head, feeling sick, fainting, and dizziness.

The measure of restlessness is evaluated with five items: feeling anxious, feeling depressed, feeling nervous, feeling irritated, and feeling annoyed.

The measure of tiredness is evaluated with five items: tired arms, pain in waist, pain in hands and fingers, tired legs, and stiff shoulders.

The measure of blurriness is evaluated with five items: eye blurring, eyestrain, eye pain, dry eyes, and blurred vision.

The five fatigue types are unevenly distributed depending on, for example, the type of work of the user or the personality of the user. Fatigue type classification estimation results are repeatedly obtained and accumulated in the storage unit 2000. The frequency of occurrence of each estimation result within a certain period of time is calculated to know results unique to the user. As a result, the feeling of satisfaction and the estimation accuracy are increased. Further, the contribution rates of the fatigue types may be calculated and the fatigue type having the highest contribution rate may be determined.

The five fatigue types change as appropriate depending on the state of the user. Identifying, for example, the correlation between the content of the task of the user and each of the fatigue types helps increase the work performance. It is also valuable to inform the user of the results at the optimal time to the user. It is also possible to make a determination in accordance with a threshold set in advance in the input information DB 2002 and notify the user of the determination.

### Representation of Fatigue Level and Concentration Level

The fatigue level and the concentration level are quantitatively evaluated. The fatigue level is a total indicator of the above-described multiple fatigue types and is easy for the user to determine his/her fatigue based thereon. Quantifying the concentration level facilitates the understanding of the influence of the environment or the content of the task. The five fatigue types are also used for fine adjustment of calculation of the remaining energy level, which will be described later, but the fatigue level and the concentration level greatly contribute to the calculation of the remaining energy level.

### Feature Value Extraction

The detector 4 extracts feature values from detected information such as the user's respiration and quantifies items of the feature values. Respiratory information, which is an example of biological information, will be described. The feature values extracted by the detector 4 are not limited to those derived from respiration, and may be feature values of various features other than respiration, such as body temperature and pulse. The feature values derived from respiration are obtained from a respiratory waveform and include, but are not limited to, a respiration rate, a respiration intensity, a respiratory waveform shape, and a respiratory variation. The feature values are acquired as appropriate and are accumulated in the storage unit 45. The processing described above is repeatedly performed at set timings or with set frequencies.

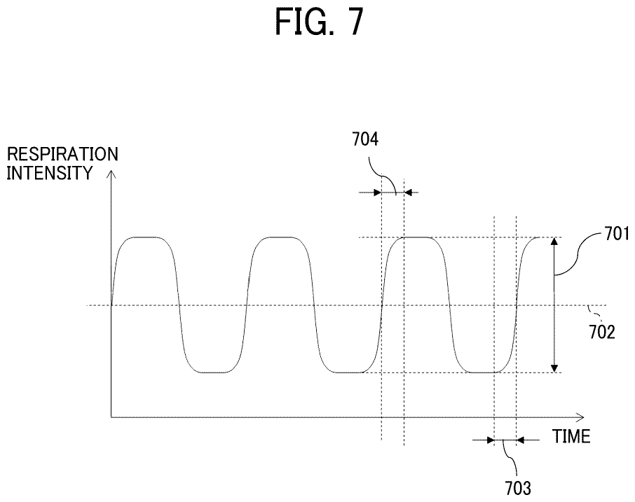

FIG. 7 illustrates an example of a respiratory waveform. The respiratory waveform is a waveform indicating a change in respiratory signal over time. The detector 4 includes an internal acceleration sensor, and can generate a respiratory waveform from the movement of the chest. The respiratory waveform is a periodic waveform and is broadly divided into three periods of time: an exhalation period, an inhalation period, and a period during which breathing stops.

Many feature values are available for respiration. Some of the feature values will be described below. One of the feature values is the time taken for breathing. The time taken for a single breath is the sum of the exhalation time, the inhalation time, and the time during which breathing stops.

The respiration rate indicates the peak-to-peak interval of the respiratory waveform, and a shorter interval indicates a higher respiration rate. The respiration intensity indicates the height of a peak of the respiratory waveform, and a higher peak indicates a higher respiration intensity. In FIG. 7, arrow 701 indicates the width of the waveform and indicates the respiration intensity.

The shape of the respiratory waveform indicates an inhalation-dominant respiratory state or an exhalation-dominant respiratory state. The balance between exhalation and inhalation is referred to as an inhalation-to-exhalation ratio, which is also effective as a feature value. The inhalation-to-exhalation ratio is quantified by individually integrating the variations in exhalation and inhalation from the average value of the respiratory waveform and recognizing the balance between the integrated variations.

The respiratory variation refers to temporal variation that occurs over a peak-to-peak interval measured on the respiratory waveform.

The feature values obtained from the respiratory variation are obtained by performing time domain analysis and frequency domain analysis on information on the respiratory variation. For example, the time domain analysis includes statistical analysis and geometric analysis such as Poincare plot analysis. The frequency domain analysis may be performed by using, for example, the fast Fourier transform (FFT) or the maximum entropy method (MEM).

The items described above are examples, but not limitation, of the feature values of respiration.

For example, the ratio of arrows 704 and 703 illustrated in FIG. 7 may also be used as a feature value of the respiratory waveform. The processing unit 43 calculates these feature values from respiratory waveforms accumulated for a set period of time, and stores the numerical values of the calculated feature values in the storage unit 45. The transmitting and receiving unit 42 transmits time-series information of the recorded feature values to the terminal device 3 and the information analysis device 2 at set intervals of time. In the present embodiment, the MPU 401 of the detector 4 performs computation, and the computation result is stored in the RAM 402. In another embodiment, the respiratory waveform is transmitted to the terminal device 3 or the information analysis device 2, and feature value extraction is performed using the CPU 301 of the terminal device 3 or the CPU 201 of the information analysis device 2.

### Estimation Method

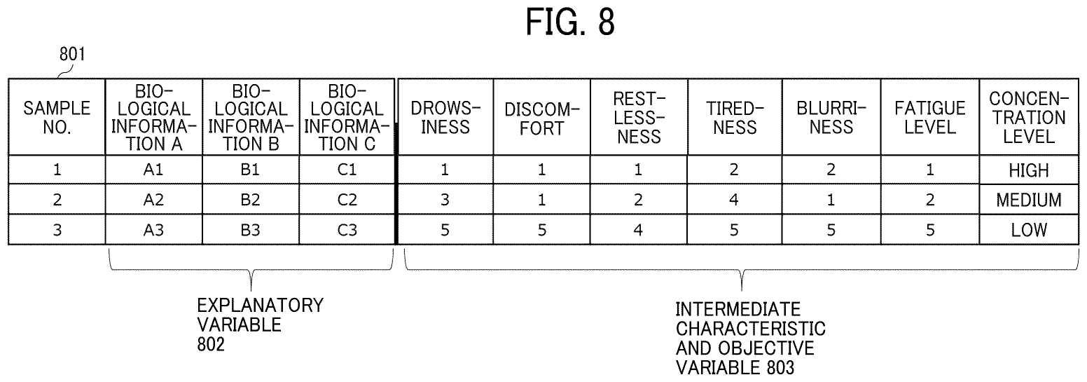

Next, a method for estimating a fatigue type, a fatigue level, and a concentration level will be described. FIG. 8 illustrates an example of datasets used for generating an estimation model. Samples with sample numbers 1 to 3 are each depicted as one row. The leftmost column indicates a sample number 801. The next three columns indicate explanatory variables 802 representing feature values of biological information, such as the respiratory information described above. The estimation model is an example of the information stored in the learned model DB 2003.

The biological information included in the training datasets includes, for example, respiratory information related to human respiration affected by a change in fatigue state or concentration state, and indicates, for example, the feature values of respiration described above.

The seven columns of fifth to eleventh columns from the left in FIG. 8 are five columns of fatigue types plus two columns of the fatigue level and the concentration level, which are objective variables 803. One example of the fatigue level or the concentration level included in the learned model is information indicating the concentration level or the feeling of fatigue, which is a subjective indicator felt by a user. Such a subjective indicator is acquired from, for example, a questionnaire. Information on a subjective indicator is collected using a numerical rating scale (NRS) or a Likert scale.

The NRS is a method used in clinical settings to evaluate, for example, “pain” severity. Specifically, the NRS is a method by which the current concentration level is rated on 0 to 10 scales, where 0 represents no concentration and 10 represents the most concentrated experience that the user has had, with 1 to 3 evaluated as low levels of concentration, 4 to 6 evaluated as moderate levels of concentration, and 7 to 10 evaluated as high levels of concentration.

The information processing system 1 according to the present embodiment includes a learned model (a learned model stored in the learned model DB 2003; the learned model may be hereinafter referred to as the learned model 2003) generated, based on subjective indicators of mental fatigue or concentration input in advance, by learning biological information and the subjective indicators of mental fatigue or concentration, the acquisition unit 25 that acquires information including biological information of a user, and the display control unit 37 that controls the display 308 to display a subjective indicator of mental fatigue or concentration determined based on the acquired information and the learned model 2003. The subjective indicators correspond to the objective variables 803 illustrated in FIG. 8. The objective variables 803 are obtained by quantifying subjective indicators felt by the user, namely, “drowsiness,” “discomfort,” “restlessness,” “tiredness,” “blurriness,” “fatigue,” and “concentration.” Accordingly, a mental fatigue indicator or a concentration level indicator, which is based on the subjectivity of the user, is estimated and output, and the output result is satisfactory to the user. Thus, the discomfort felt by the user for the output results is reduced. The visualization of the concentration level of the user allows the user to take an action of increasing the concentration level. The reduced discomfort reduces the stress felt by the user when the user uses the information processing system 1, and motivates the user to continuously use the information processing system 1.

The datasets for training, which are created in the way described above, are used to generate an estimation model. While three samples are illustrated in FIG. 8, a large number of samples are used in actuality.

While three columns of feature values are illustrated in FIG. 8, more feature values may be used. The use of a large amount of information may increase the estimation accuracy. On the other hand, such a large amount of information may lead to noise, resulting in reduced estimation accuracy. It is therefore desirable to select an appropriate number of samples and an appropriate number of items.

In one embodiment, the learning algorithm is a statistical causal inference algorithm. In another embodiment, a known algorithm such as supervised learning, unsupervised learning, or reinforcement learning may be used. Statistical causal inference is a technique for estimating a causal structure and a causal parameter between an explanatory variable and an objective variable from data.

Supervised learning is a method for providing a combination of input data and correct output data corresponding to the input data as training data and learning relationship between the input data and the output data.

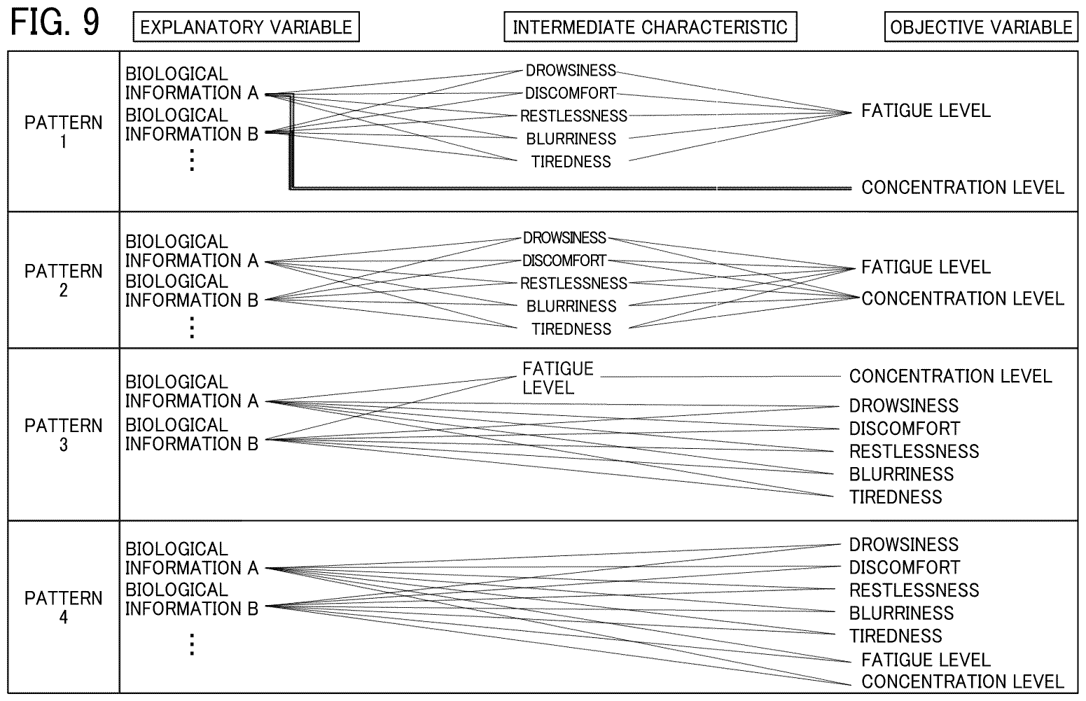

Methods of machine learning are illustrated in FIG. 9. The methods are denoted by patterns 1 to 4. The methods use different objective variables and different explanatory variables.

Unsupervised learning is a method for learning features included in input data without providing training data. Pattern 1 is a typical example of unsupervised learning. In pattern 1, the fatigue level and the concentration level are set as objective variables, and the fatigue types are set as intermediate characteristics of the fatigue level, thereby making it possible to grasp the fatigue type that mainly causes the fatigue level to recommend an effective recovery action. In pattern 2, the fatigue level and the concentration level are set as objective variables, and the fatigue types are set as intermediate characteristics, thereby making it possible to grasp an issue that distracts the user from concentrating on their task. In pattern 3, when the concentration level is set as an objective variable, the fatigue level is set as an intermediate characteristic. In pattern 4, the fatigue level, the concentration level, and the fatigue types are set as objective variables.

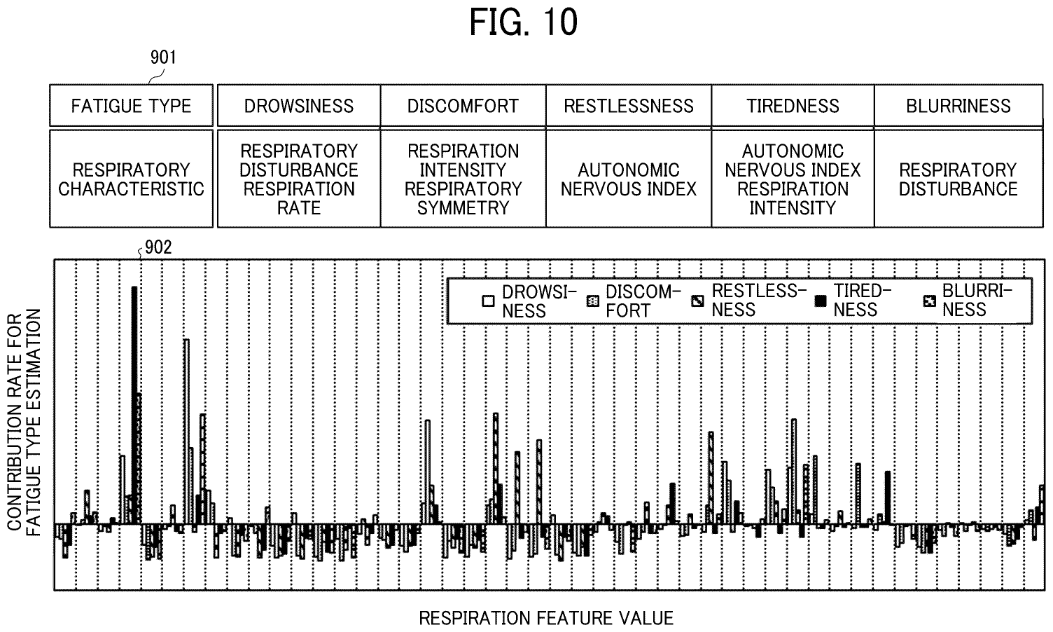

FIG. 10 illustrates the feature values of respiration and the contribution rates for fatigue type estimation. The contribution rates of five items, which are the fatigue types, are provided with respect to more than 40 feature values of the respiratory waveform plotted on the horizontal axis. The fatigue types refer to the five types described above, namely, “drowsiness,” “discomfort,” “restlessness,” “tiredness,” and “blurriness.” As illustrated in FIG. 10, feature values exhibit the presence of high correlations between the fatigue types and the respiratory information indicating the respiration of a person.

For example, the fourth item from the left indicates relatively high contribution rates, and, in particular, indicates the highest contribution rate for “tiredness.” The presence of such correlations is known, and several studies have been conducted. For example, with increasing drowsiness, the disturbance of respiration increases, leading to a decrease in respiration rate. In contrast, discomfort is a feature value indicating the most significant correlation with the disturbance of respiration. Each fatigue type has a different feature value with a high correlation. Due to this difference in feature value, each fatigue type has different contribution rates of the feature values. In terms of such differences, an estimation model is constructed. In one embodiment, these differences are used to estimate a fatigue type from the feature values obtained from the biological information. The estimation model is constructed by the machine learning described above, and an item indicating a plurality of feature values with high contribution rates, as illustrated in FIG. 10, can be estimated with a high probability.

Feature value analysis results that are repeatedly obtained and the learned model 2003 stored in the storage unit 2000 are used to estimate the strength of fatigue. For example, the learned model 2003 is a machine-learned model that has learned in advance changes in the feature values of biological information in accordance with changes in fatigue score. Repeatedly obtained estimation results are accumulated in the storage unit 2000, and an average value of the estimation results within a certain period of time is calculated to estimate the fatigue levels of two or more classes. Further, the concentration levels of two or more classes are estimated using a similar method.

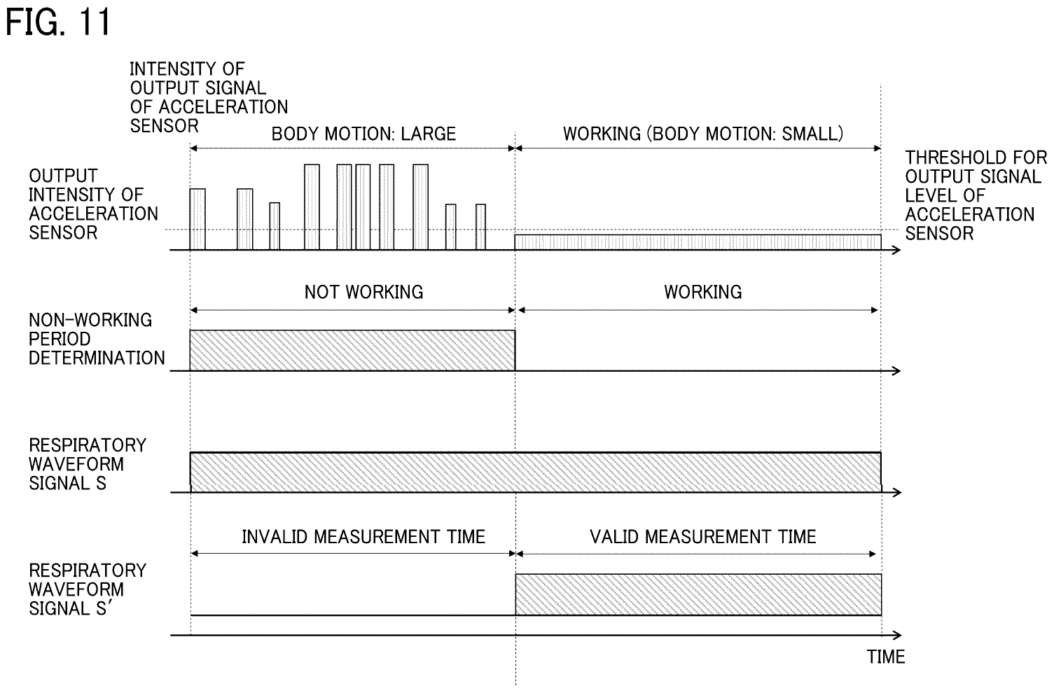

Next, a method for measuring a high-quality respiratory waveform will be described. FIG. 11 illustrates determination of seating. It is found that the respiratory waveform includes much noise when body motion is large or the user leaves the seat. In FIG. 11, the body motion of the user is detected based on the output intensity of the acceleration sensor or the like included in the inertial measurement unit. The computation unit 27 estimates the state of the user who is seated and working so that the accuracy of estimating, for example, the fatigue and the concentration of the user is not compromised.

A threshold for the output signal level of the acceleration sensor is set, and it is determined that body motion is large when the signal intensity continues to exceed the threshold. For a respiratory waveform signal, only the period of time during which it is determined that the user is working is used as a valid measurement time. As described above, measuring only the period of time in which it is determined that the user is performing work in a seated state at rest enables the acquisition of an accurate respiratory waveform signal. Accordingly, the estimation accuracy is not compromised.

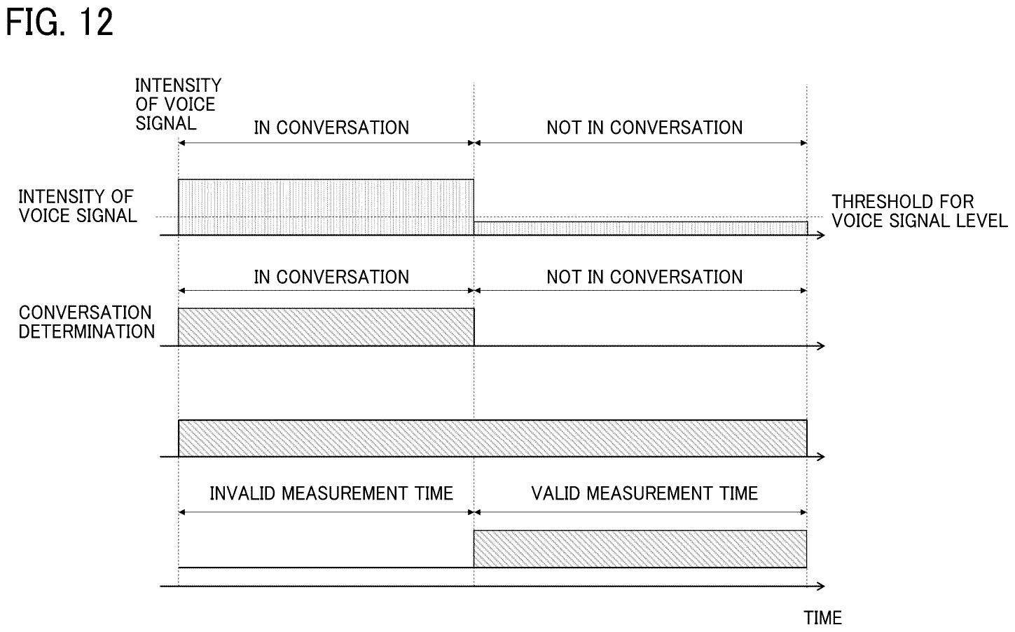

FIG. 12 illustrates a conversation determination method. It is found that the respiratory waveform includes noise when the user has a conversation. The conversation determination method is intended to detect a conversation that the user has with someone in accordance with the output intensity of a voice signal from the microphone, and estimate the state in which the user is in conversation such that the accuracy of estimating, for example, the fatigue state and the concentration state of the user is not compromised.

A threshold for the output signal level of a voice signal from the microphone is set, and it is determined that the user is in conversation when the signal intensity continues to exceed the threshold. For a respiratory waveform signal, a period of time during which it is determined that the user is not in conversation is used as a valid measurement time. As described above, measuring only the period of time in which it is determined that the user is performing work at rest without conversation enables the acquisition of an accurate respiratory waveform signal. Accordingly, the estimation accuracy is not compromised.

### Visualization Method

### Visualization of Concentration Level, Fatigue Level, and Fatigue Types

Next, items to be visualized will be described. An example of the visualization is illustrated in FIG. 6. The display screen 600 displayed on a computer presents the concentration score 601, the fatigue score 602, and the radar chart 603 representing the fatigue types. The fatigue types may be represented by a radar chart as illustrated in FIG. 6 or may be represented in a visually appealing manner such as using colors. The radar chart has an advantage in that, since the fatigue types as the breakdown of the fatigue level are presented, the viewer can understand the balance or characteristics in the composition ratio while grasping the respective magnitudes of the different types of fatigue.

As illustrated in FIG. 6, the estimation results of the feeling of fatigue and the concentration level, which are obtained by the computation unit 27, may be digitized and displayed as relative scores. Scoring the results and displaying the scores of the results in an at-a-glance manner allow the user to grasp the state of the user in real time. In addition, the five fatigue factors are also presented to the user, thereby reducing the discrepancy from the user's own sensation and increasing the satisfaction with the visualized results.

The information processing system 1 according to the present embodiment includes the acquisition unit 25 that acquires information including biological information of a user (step S14), the learned model 2003 that has learned the relation between biological information acquired in advance and types of mental fatigue, and the display control unit 37 that displays on the display 308 (step S30) a type of mental fatigue determined (step S18) based on the acquired information and the learned model 2003. As illustrated in FIG. 6, the display screen 600 displays fatigue types in a radar chart. Accordingly, a type of mental fatigue of the user is estimated and output, and the output result is satisfactory to the user. Thus, the discomfort felt by the user for the output results is reduced. The visualization of a fatigue type allows the user to take an action that prompts recovery.

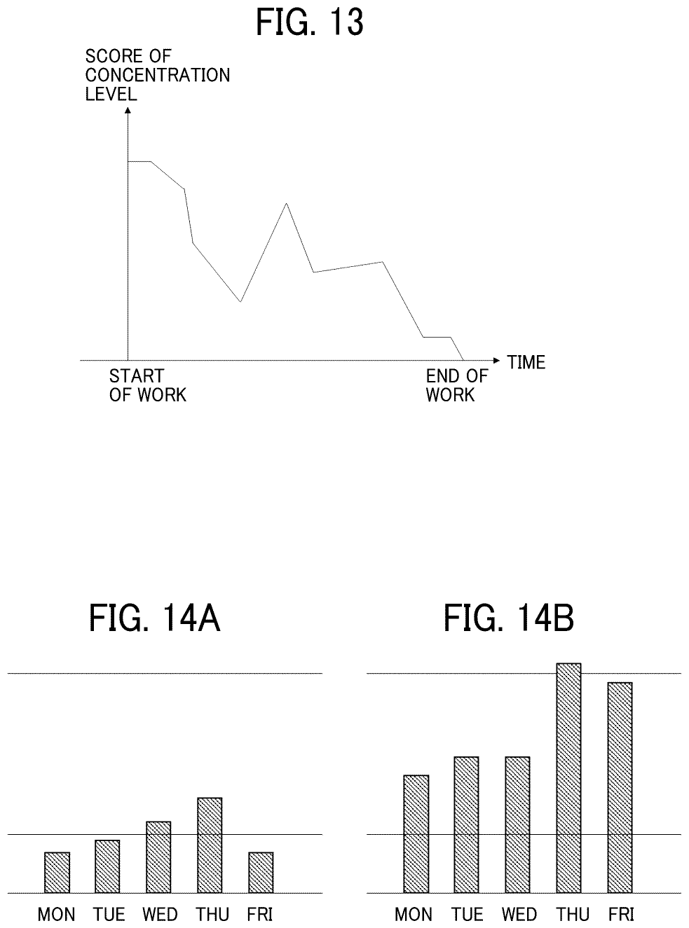

As illustrated in FIG. 13, the estimation results of the fatigue level and the concentration level, which are obtained by the computation unit 27, may be converted into a graph over time, and the graph may be output to be displayed. Displaying the results with time plotted on the horizontal axis allows the user to grasp a point of state change in a day at a glance. A point of state change of a user in a day is associated with an action of the user to provide an opportunity for the user to review his/her work.

As illustrated in FIGS. 14A and 14B, average daily scores of the fatigue level or the concentration level, which are obtained by the computation unit 27, may be indicated by a bar graph. Displaying a list of scores before and after the start of work in a week allows the user to grasp a change in state over the week. Since the user can grasp at a glance a tendency for a change in his/her state over a week, the change tendency over the week and actions of the user are objectively associated with each other to provide an opportunity for the user to subjectively review their work.

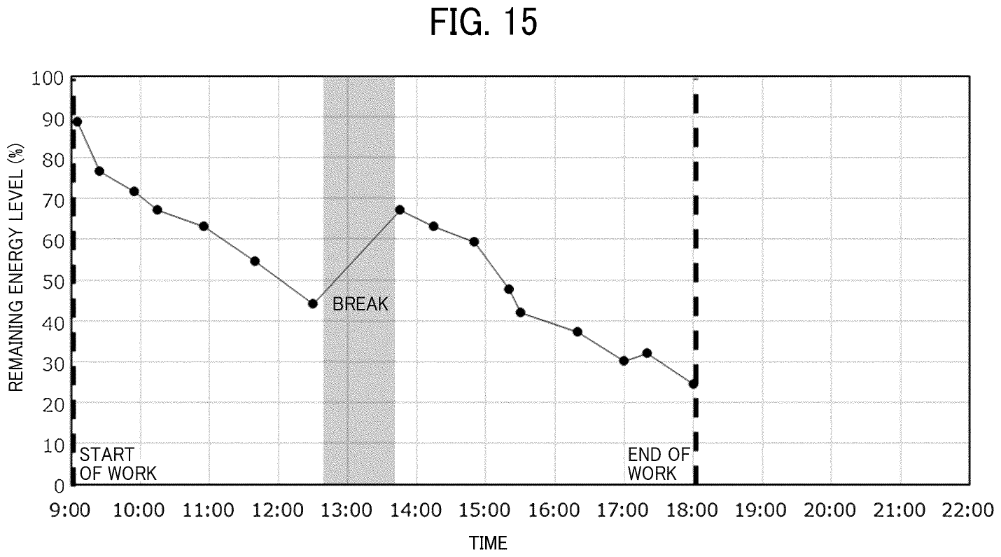

As illustrated in FIG. 15, estimated values of the fatigue level may be converted into an indicator of the remaining energy level to more clearly display the estimated values to the user. The remaining energy level is a concept created in the present embodiment and is found to be highly appealing. The remaining energy level is a representation of finite energy possessed by a person in a manner similar to that for a battery or the like of a mobile phone. Any representation or calculation method that allows the user to easily understand estimated values of the fatigue level may be used.

The estimated values of the fatigue level caused by tasks, meetings, and other factors increase with time. Accordingly, remaining energy levels are calculated as the inverse of the estimated values of the fatigue level. The remaining energy level is calculated with respect to reference values, with the reference values being 90% at the highest estimated value of the fatigue level for a reference time period and 30% at the lowest estimated value of the fatigue level for the reference time period. The reference time period may be one week from a time when the information processing system 1 is first used, or a time period from the latest one week to the latest one month.

The reference values may be determined with reference to estimated values for the user, or may be prepared by the system administrator. When fatigue occurs, the estimated value of the fatigue level increases, resulting in a decrease in remaining energy level. When the user recovers from the fatigue by performing a recovery action, the estimated value of the fatigue level decreases, resulting in an increase in remaining energy level. Displaying the remaining energy level of the user in percentage in the way described above allows the user to associate their actions with fatigue levels at a glance.

It has been found that recovery is significantly degraded when the remaining energy is used up to 0%. It is therefore desirable that the user have a remaining energy level of, for example, 20% before a break such as weekend starts. However, it is difficult to leave a certain amount of remaining energy as appropriate in accordance with the user's subjective determination. The user who experiences the subjective feeling of fatigue may fail to make an appropriate decision at an appropriate timing. It is therefore desirable that the information processing system 1 external to the user determine the subjective feeling of fatigue that the user experiences and provide a message based on a quantitative determination made by the information processing apparatus. Accordingly, in one embodiment, the user re-recognizes their subjective sensation and takes an appropriate action.

The information processing system 1 according to the present embodiment estimates the remaining energy level of the user based on the subjective indicator of mental fatigue and the type of mental fatigue (step S25). This configuration makes the remaining energy level visible to the user, making it easier for the user to take the next action, such as taking a break or entering a concentration mode. For example, comparing the state of a user at the start of work on each day with the state of the user at the start of daily work in the past or considering previous behaviors that the user exhibits from various aspects allows the user to easily identify the cause of a disorder experienced by the user or the cause of fatigue experienced by the user or to be easily aware of their unique tendency for disorders or fatigue. Accordingly, the user can easily find a recovery action suitable for the user, maintain health, and maintain performance. In addition, the user can optimize weekly scheduling for tasks by themselves.

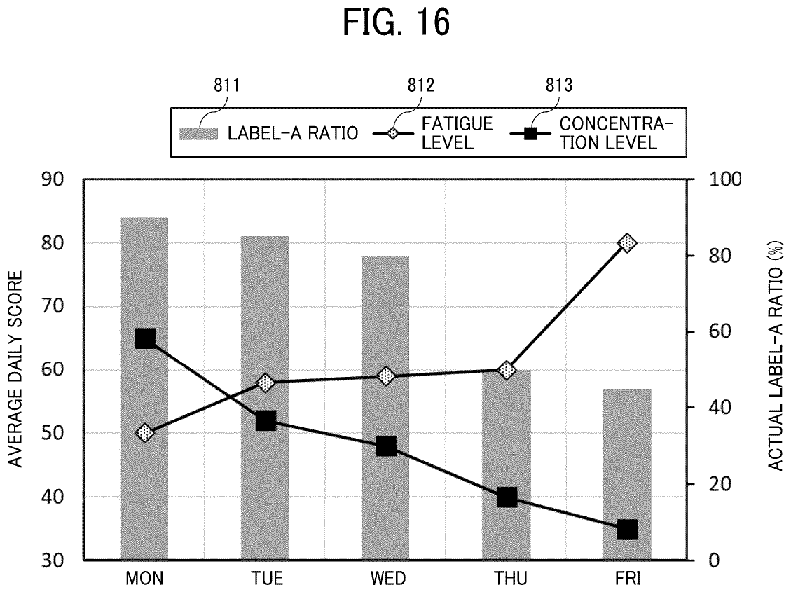

FIG. 16 illustrates an example of fatigue levels and concentration levels displayed on a weekly basis. Lines 812 and 813 respectively represent day-by-day average scores of fatigue and concentration in a week. The illustrated example is obtained by calculation from data accumulated on a several-month basis. In another embodiment, results over a long period of time, such as on a yearly basis, may be used. In another embodiment, the percentages of the average daily scores of concentration levels and fatigue levels and actual work performance may be displayed simultaneously in a graph. The user can grasp a tendency for a change in actual work performance and the state of the user at a glance, and the change tendency over a week and actions of the user are associated with each other, thereby providing an opportunity for the user to review their work. At this time, one of the fatigue types may be selected as a label-A ratio 811, and the change of the selected fatigue type over time may be observed. A label represents one of the five fatigue types described above and is, for example, “drowsiness.” A fatigue type, such as “drowsiness,” displayed on a weekly basis may exhibit apparent periodicity. Examples of the periodicity include a difference between the start of the workweek and the weekend.

The information described above can help the user take measures or manage a schedule.

### Second Embodiment

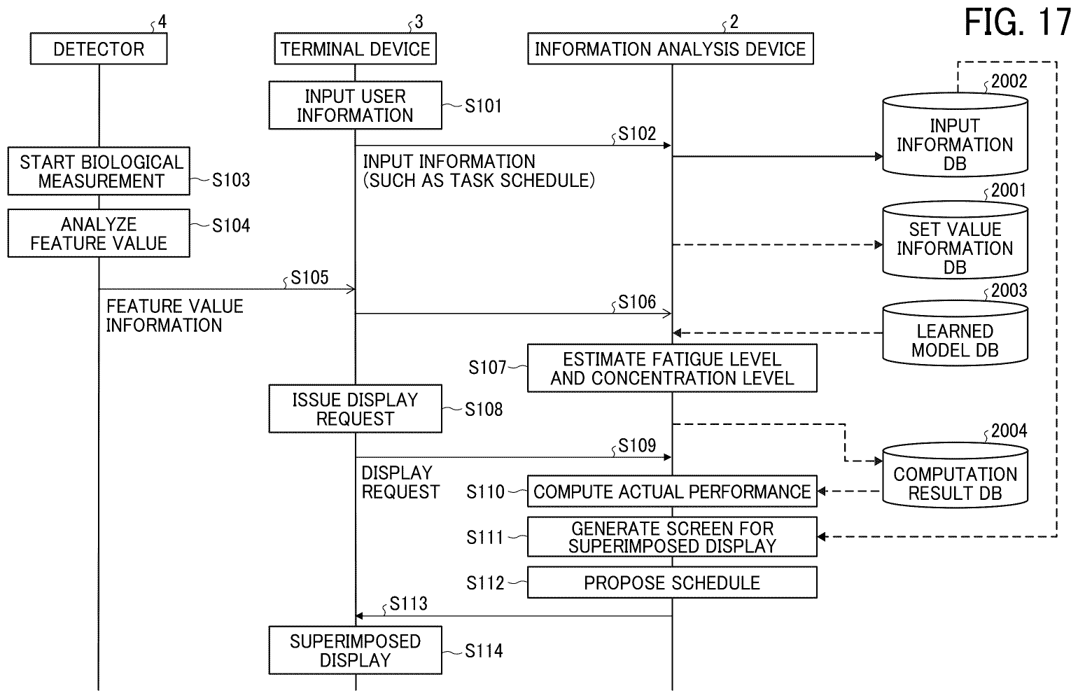

Next, a second embodiment will be described. A feature of the second embodiment is that a user's tendency for the concentration level (fatigue level or fatigue type) is displayed to be superimposed on a schedule table to make an efficient and optimal schedule. The schedule table is an example of schedule information. The above feature is based on the fact that, as illustrated in FIG. 16, long-term data accumulation allows periodic variations on a weekly basis to appear as a tendency of the user and such periodic variations can be visualized. The tendency of the user is also the unique personality of the user. FIG. 17 is a sequence diagram of a process for assisting in optimizing a schedule according to the present embodiment.

While the following description will be given of the concentration level, the fatigue level and the fatigue types may also be represented in a superimposed manner. In particular, the fatigue types are likely to significantly reflect the personalities of users, and schedule management tailored to each individual user is effective. For example, the “tiredness” is affected by blood pressure fluctuations that undergo circadian rhythm, and a person with anemia feels more tired in the morning. Further, the “drowsiness” of a person with a big appetite increases, for example, after a meal because of a reduction in the cerebral blood flow or the postprandial blood glucose level, which is affected by the amount of insulin. Such reactions may depend on the physical personality of the user or the way in which the user cats a meal.

First, the user inputs information such as a schedule. The information includes information directly input by the user via the input unit 35, such as a scheduled work start time, a scheduled work end time, and a desired work time, information input on the PC screen by the user using the keyboard 311, information selected by the user through a mouse operation from within displayed information, and information (such as scheduled information of a calendar application) acquired from another application used by the user using an API (step S101). The input information is transmitted to the information analysis device 2 and is recorded in the input information DB 2002 (step S102).

The detection unit 44 of the detector 4 starts measuring the biological information of the user (step S103). The detector 4 analyzes feature values from the input biological information (step S104), and transmits information on the analyzed feature values to the terminal device 3, as appropriate (step S105). The information is also transmitted to the information analysis device 2 (step S106).

The computation unit 27 of the information analysis device 2 analyzes the information in the learned model DB 2003 and the transmitted feature values and estimates a fatigue level, a concentration level, and a fatigue type (step S107). The estimated results are recorded in the computation result DB 2004, as appropriate.

The user issues a display request from the terminal device 3 (step S108). The display request also includes a request to display the schedule table and the concentration level (fatigue level or fatigue type) in a superimposed manner. In one embodiment, an optimum schedule proposal may be requested. Information including the type of the request is transmitted to the information analysis device 2 (step S109).

Upon receipt of the request, the information analysis device 2 reads the past history from the computation result DB 2004 and computes the actual performance from the past history (step S110). Through the computation, a result reflecting, for example, the tendency of the user is calculated. The information analysis device 2 generates a visualized display of the tendency of the user. The computation unit 27 of the information analysis device 2 generates a display screen to display a diagram indicating the tendency for the concentration level (fatigue level or fatigue type) and the schedule in a superimposed manner (step S111). While the description is given of the concentration level, in one embodiment, the tendency of the user may be expressed by, for example, the fatigue level or the fatigue type. In particular, the fatigue type is likely to reflect the personality of the user, and has an advantage in that the satisfaction of the user increases.

In response to a request, the information analysis device 2 proposes an optimal schedule (step S112). In one example, the concentration level (fatigue level or fatigue type) tends to increase in the morning in the first half of the week. In this case, the information analysis device 2 proposes that a high-difficulty task with results be executed in that time slot. The generated display screen is transmitted to the terminal device 3 (step S113) and is displayed by the display 308 of the terminal device 3 (step S114).

### Method for Superimposed Representation of Schedule and Concentration Level (Fatigue Level or Fatigue Type)

Next, a method for superimposed representation of the schedule and the concentration level (fatigue level or fatigue type) will be described. The terminal device 3 generates a schedule table using the input information (step S101) entered by the user. The input information includes tasks and time slots allocated to the tasks. The tasks include, for example, time information such as an idle time, a meeting time, a work time, a break time, a work start time, and a work end time.

The idle time is a time that the user is allowed to allocate to their work.

The meeting time is a time taken for attendance at a meeting or a lecture, which is either online or offline or is set by another person. The work time is a time taken for the user to work. The break time is a time during which the user can take a meal or take a break to refresh. The work to be scheduled is a task set by the user themselves, and examples of such a task include a document creation task and a programming task. The management of the respective times is desirably performed on a 15-minute basis. In one embodiment, the respective times may be set in increments of 1 minute.

Then, the history of the concentration level (fatigue level or fatigue type) is calculated. The history of the concentration level (fatigue level or fatigue type) can be calculated on any time axis such as that illustrated in FIG. 13, 14A, 14B, or 15. From the history of the concentration level, a tendency unique to the user is obtained. The user's unique tendency for the concentration level is displayed in the schedule table.

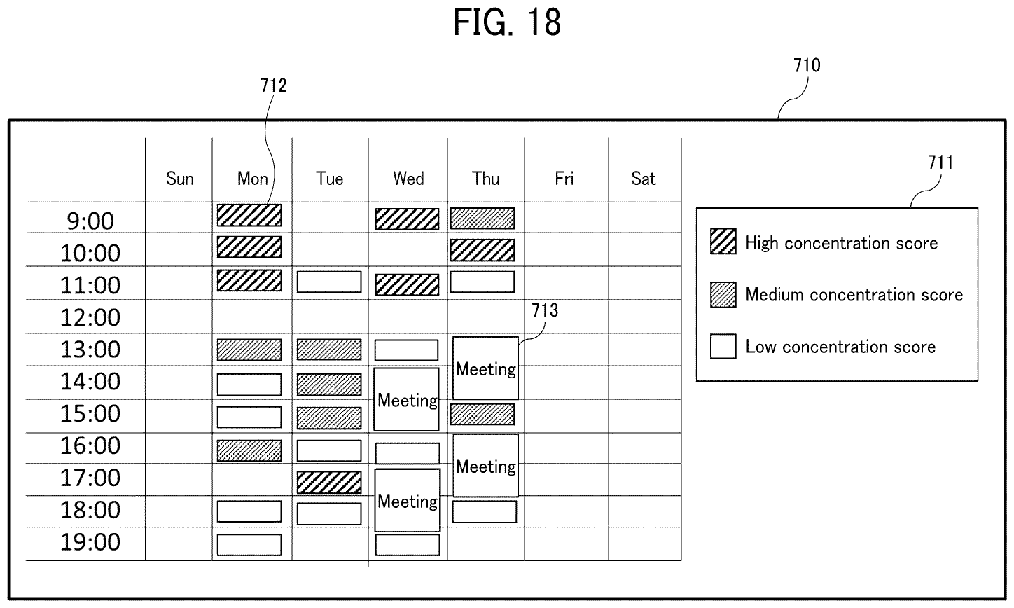

FIG. 18 illustrates an example of a schedule table 710 on which a concentration level 712 is displayed in a superimposed manner. The schedule table displays tasks such as a meeting 713, as described above, in accordance with the respective entered times. The schedule table 710 illustrated in FIG. 18 is a weekly timetable. The concentration levels (fatigue levels or fatigue types) are scored and presented in the timetable to allow the user to understand weekly scheduled tasks at a glance. In particular, since the timetable includes holidays, the tendency for the concentration level (fatigue level or fatigue type) before and after the holidays reflects a tendency unique to the user. Three levels of concentration scores 711 are displayed. The three levels of concentration scores 711 may be expressed by a visually recognizable method such as in colors. In one embodiment, scores digitized for case of understanding may be used. Accordingly, the user viewing the output results displayed on their terminal can grasp, at a glance, a tendency for a change in the state of the concentration level (fatigue level or fatigue type) associated with the actual performance. The output results may be displayed on an hourly basis or on a 15-minute basis.

The schedule table allows the user to find idle times. Among the idle times, a time slot in which the concentration level (fatigue level or fatigue type) tends to be high is visualized for clear understanding. This allows the user to determine in which time slot to process a new task to efficiently schedule the new task. In addition, the user can identify a tendency for the concentration level (fatigue level or fatigue type), and can allocate an appropriate task to an appropriate time slot.

### Method for Proposing Schedule

In the present embodiment, when a new task is to be scheduled, an appropriate time slot and an appropriate schedule can be proposed. A method for proposing such a schedule will now be described.

In the sequence diagram illustrated in FIG. 17, in the input of user information (step S101), the actual performance of the user in the previous processing of tasks are input.

The actual performance of the user refers to a meeting, a break, or work that has progressed as scheduled and the content thereof, and a meeting, a break, or work that has not progressed as scheduled and the content thereof. The time taken for a task that has progressed as scheduled to complete and the time taken for a task that has not progressed as scheduled to complete are calculated by using the information input using the input unit 35.

A list of schedules for the past one day or the past one week is displayed, and the user inputs actual performance information for each of the schedules. The actual performance information includes four types of classification information, namely, labels A, B, C, and D, and information on implementation items such as achievement levels.

The user inputs, as the actual performance information, the achievement level of each schedule, indicating the degree to which the schedule has been achieved. The label A is assigned to a schedule that has been completed on time. The label B is assigned to a schedule that has been completed after the scheduled end time. The label C is assigned to a schedule that has not been completed. The label D is assigned to a schedule when a different schedule has been performed instead of the schedule. For a schedule to which the label D is assigned, information on a reason for non-completion and the actually implemented event are provided as actual performance information and are accumulated in the input information DB 2002 of the storage unit 2000.

In addition, a digitized indicator is recorded as the achievement level to help understand the level of progress. An achievement level of 100% is set for the labels A and B. For the label C, the corresponding achievement level is recorded. An achievement level of 0% is recorded for the label D.

Actual performance for the “user's schedules” is calculated for each label. For example, each of the pieces of actual performance information with the labels A, B, C, and D assigned is subjected to the following process. A work time, a meeting time, and a break time are calculated from actual performance information between a work start time and a work end time and are accumulated in the storage unit 2000. The sum of work times, meeting times, and break times in one week, and the sum of work times in each day are calculated and accumulated in the input information DB 2002 of the storage unit 2000. This allows the user to easily grasp how much time the user spends on the schedules.

Then, the terminal device 3 analyzes the actual performance of the user by using the input information (step S101). A difference in actual performance from the “user's schedules” is calculated for each label and each schedule. A difference between an actual time and a scheduled time from the work start time to the work end time is calculated and accumulated in the input information DB 2002 of the storage unit 2000. The transition of the time difference from the schedules for one week is accumulated in the storage unit 2000. This allows the user to easily grasp whether the user is good at or bad at each schedule.

Then, schedule optimization computation is performed. An optimized schedule is computed using the schedule information, the actual performance information, and the input information of the user. A work time and a break time may be automatically arranged in an idle time in the user's schedules so that a desired work time set by the user is exceeded.

The computation process may include a method for allocating a work time by utilizing the actual performance information accumulated in the storage unit 2000. For example, if a task equivalent to a task that has not progressed as scheduled is registered in the user's schedules, the time to be taken for the task to complete can be increased from the actual performance information accumulated in the input information DB 2002 and can be allocated to the task. The schedule computation results are accumulated in the computation result DB 2004 of the storage unit 2000, and a proposed schedule is presented to the user on the display screen illustrated in FIG. 18. At this time, a schedule of a task may be proposed in consideration of a time slot in which the concentration level (fatigue level or fatigue type) is high and the content of the task. In this case, the feasibility of a task is calculated taking into account a time constant determined for the task, which will be described below, and a schedule is proposed so that the task is scheduled in a time slot with appropriate feasibility.

The user may approve the proposed schedule, or may change the proposed schedule.

A modification of the schedule table using a function of another application (an application having an existing calendar function) will be described. Information acquired from another application used by a user using an API is useful in a wide variety of situations. For example, there is another application having a function of easily setting a “focus time” by automatic setting or repetitive setting. The information processing system 1 may further has a function of adjusting the start time, the end time, and the length of the focus time set in the application in accordance with the concentration level 712 (fatigue level or fatigue type) obtained by the information processing system 1, or issuing proposal or warning about the focus time. Accordingly, the information processing system 1 performs fine adjustment and optimization of a schedule that is easily input using another application, thereby increasing the work efficiency of the user.

For example, the date and time of the meeting 713 and the length of the time for the meeting 713 may be finely adjusted by a person who arranges the meeting 713, based on the information on the concentration level 712 of a user who participates in the meeting 713. In the input of user information (step S101), users who participate in a meeting are identified, and data of the users is collected from the computation result DB 2004. The information analysis device 2 performs actual performance computation (step S110) of the users to calculate optimum conditions for most of the users. The information analysis device 2 performs schedule proposal (step S112) such that the conditions described above are satisfied. At this time, users who participate in the meeting may be identified, and information on the schedules of the users who participate in the meeting may also be acquired from another application via the API. In addition, the users may be notified of the result of the schedule proposal (step S112) via push notification under another application through the API.

In one embodiment, a notification function included in the functions of another application may also be used. It is also effective to, for example, change the schedule in accordance with the concentration level (fatigue level or fatigue type) at the timing of the notification. In addition, a function of, at this timing, automatically adjusting the schedule information in accordance with whether the score of the concentration level (fatigue level or fatigue type) is high, medium, or low may be provided.

In the information processing system 1 according to the present embodiment, the acquisition unit 25 that acquires information on schedules including tasks of a user, and the display control unit 37 displays, on the display 308, schedule information indicating tasks of the user for a certain period of time. Further, the display control unit 37 displays, on the display 308, the estimation result of the mental fatigue type or a subjective indicator of mental fatigue or concentration superimposed on the schedule information. This configuration provides simultaneous visualization of a tendency of the user and the schedules, allowing the user to select an appropriate time slot when scheduling the next task, and to efficiently perform the task. Since time slots in which the user tends to concentrate are identified in advance, the user schedules tasks while understanding the tendency. Thus, the user can perform the tasks in a well-motivated manner without being unnecessarily stressed. Fatigue levels or concentration levels associated with previous tasks are visualized to allow the user to identify a tendency, which provides an opportunity to reduce unnecessary work or meetings when, for example, sufficient time is not available.

### Third Embodiment

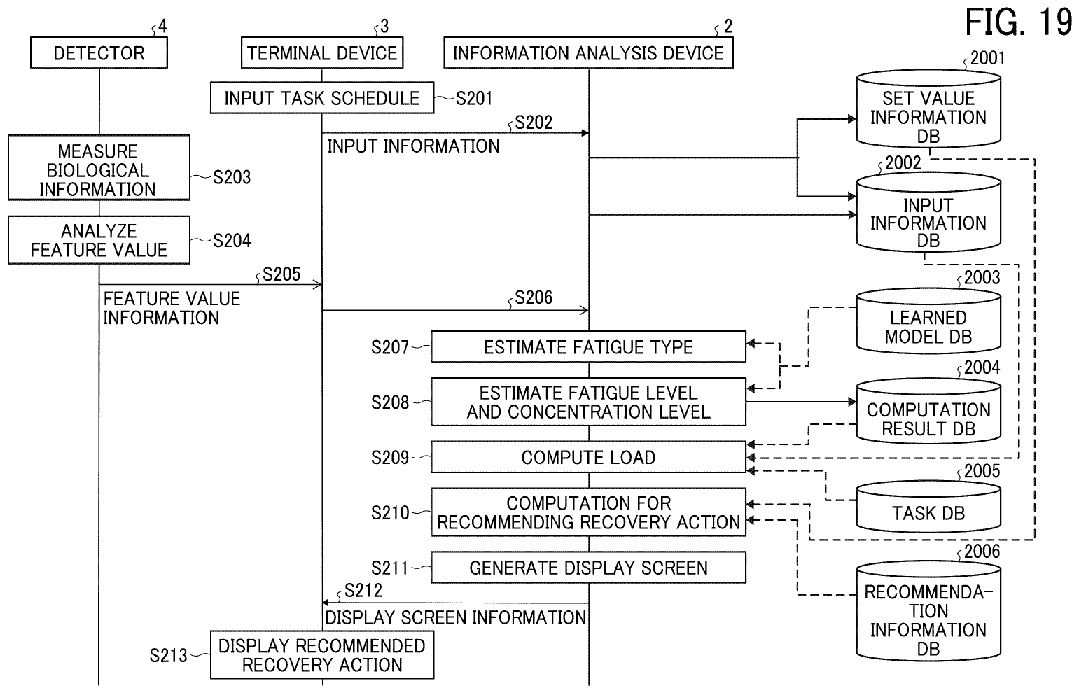

In a third embodiment, a user is notified of a recovery action at an appropriate timing from an obtained fatigue type or remaining energy level. FIG. 19 illustrates a sequence diagram.

Information related to tasks of a user is input from the terminal device 3 (step S201). The information is transmitted to the information analysis device 2 (step S202) and is recorded in the input information DB 2002. The detector 4 measures the biological information of the user (step S203) and analyzes feature values (step S204). The analysis results are transmitted to the terminal device 3 as appropriate (step S205). The analysis results are also transmitted to the information analysis device 2 (step S206). From the information on the feature values, the information analysis device 2 estimates a fatigue type (step S207) and a concentration level and a fatigue level (step S208). The results are recorded in the computation result DB 2004.

The load placed by a currently performed task is computed from the corresponding task information recorded in the input information DB 2002 (step S209). In the computation, the load is computed based on, for example, the time constant information of the task recorded in the task DB 2005. Through the computation, a change in remaining energy level, which is expected in the future, is estimated. The estimated change in remaining energy level and, as appropriate, the input biological information (step S206) are added to the computation.

The computation of the change in remaining energy level is repeated as appropriate, and it is determined whether the remaining energy level exceeds a set threshold. If the remaining energy level exceeds the set threshold, the information analysis device 2 performs computation for recommending a recovery action (step S210). An appropriate recovery action is selected from the estimated fatigue type and the estimated remaining energy level. The selection is performed using a table recorded in the recommendation information DB 2006.

The computation unit 27 of the information analysis device 2 generates a display screen (step S211) and transmits the display screen to the terminal device 3 (step S212). The terminal device 3 displays the display screen (step S213).

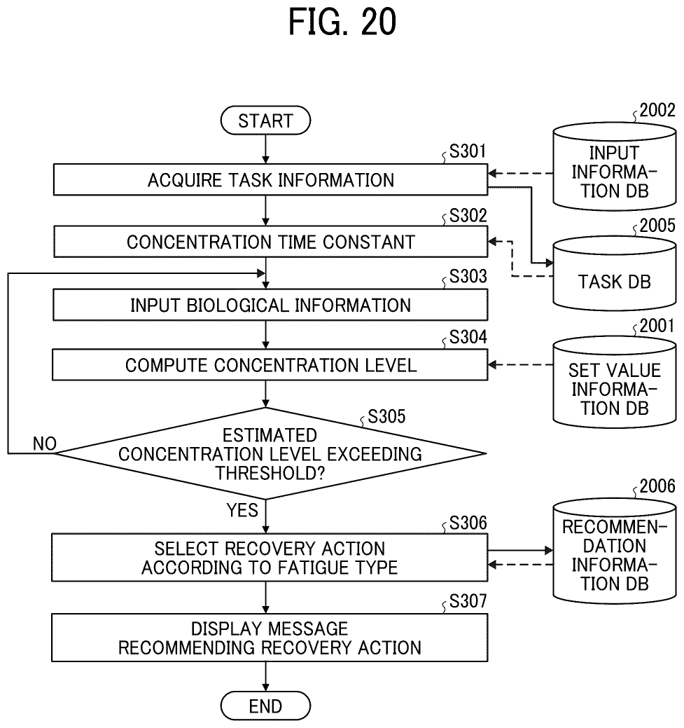

Next, the timing at which a recovery action is displayed will be described with reference to a flowchart illustrated in FIG. 20. In general, many users perform recovery actions after a remaining energy level of 0% is reached, and in this case, much time is taken for such users to recover. The remaining energy level is objectively quantified, and a recovery action is performed when some energy remains, thereby achieving efficient recovery. In the present embodiment, such an appropriate timing is notified to the user using the terminal device 3.

Task information of a task that the user is currently performing is acquired from the input information DB 2002 (step S301). Based on the task information, a corresponding task is extracted from within the information in the task DB 2005. The concentration time constant of the extracted task is calculated from the table in the task information DB (step S302).

The detector 4 receives input of biological information and extracts feature values (step S303). The detector 4 transmits information on the extracted feature values to the information analysis device 2. From the information on the feature values, the information analysis device 2 computes a fatigue type, a fatigue level, a concentration level, and a remaining energy level (step S304). At this time, the set value information DB 2001 includes the information input in step S201, and a threshold for the concentration level or the remaining energy level is set in accordance with the information. It is determined whether the threshold is exceeded (step S305). If the threshold is not exceeded, the input of biological information is continued (step S303). Further, the detector 4 continuously transmits the feature values to the information analysis device 2 as appropriate.

If the concentration level or the remaining energy level exceeds the threshold (YES in step S305), a recovery action is selected in accordance with the fatigue type (step S306). The selection is performed with reference to the table in the recommendation information DB 2006.

The information analysis device 2 generates an image of a message recommending the selected recovery action. The image is transmitted to the terminal device 3, and the display 308 of the terminal device 3 displays the image (step S307).

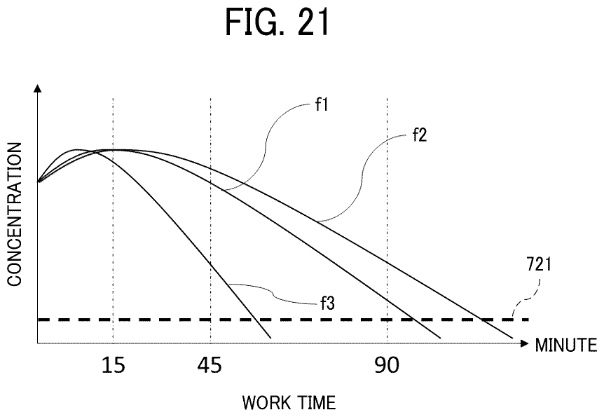

Next, a method for computing a task load will be described. FIG. 21 is a graph illustrating relationship between the work time and the concentration of a user who is concentrating on work during the work. It is difficult to concentrate for a long time, and it is desirable that a user take a break at a certain time. It is generally said that a person can concentrate for up to 90 minutes, as indicated by a curve f1, and a high-concentration task is generally segmented by time. It is considered that, for example, a person can focus on a task with high concentration, such as simultaneous interpretation, for up to 15 to 20 minutes. In contrast, normal tasks, such as attending a class lecture, generally last 45 to 50 minutes, and university classes generally last 90 minutes.

Before the concentration decreases, a task is segmented at appropriate timings to relax the concentration, thereby achieving high performance on the task as a whole. However, the duration of concentration is likely to vary from one individual to another, depending on physical conditions, or the like. As indicated by a concentration curve f2, the concentration may last longer, or, as indicated by a curve f3, concentration may decrease rapidly. Accordingly, the state or tendency of the user is grasped in real time from the biological information, thereby making it possible to display recommended recovery actions such that a task is segmented at appropriate timings.

Examples of recommendation of an action to improve the performance of daily tasks include setting a timer and performing tasks with short breaks in between. A prediction curve of concentration levels of a user from the work start time to the work end time in a certain day is computed from tendencies of the user for changes in concentration level in the past, which are accumulated in the storage unit 2000. Before the completion of a suspended task, if it is determined that the concentration level of the user is lower than a preset limit, the user is prompted to take a break. The length of the duration of a break that the user is prompted to take may be set to an appropriate value by the system in advance, or may be set by the user. As indicated by a broken line 721 illustrated in FIG. 21, a certain threshold may be set. The amount of effect of taking a break is calculated from the amount of change in concentration level or remaining energy level before and after the break, and is recorded in the storage unit 2000. In one embodiment, a concentration curve obtained when a user takes breaks and a work curve obtained when the user takes no break may be compared, and the comparison result may be output.

Accordingly, in consideration of a change in the duration of concentration from one individual to another or depending on physical conditions, actions are recommended such that a task is segmented at appropriate timings to keep the user's concentration high, thereby increasing the performance of daily tasks as a whole.

It is possible to appropriately relax the concentration in consideration of not only a change in the duration of concentration from one individual to another or depending on physical conditions but also the types of tasks, thereby supporting the user in recovery according to their work. An action for recovery is recommended in consideration of the context information of the user. Thus, the user can be notified of the most effective recovery action that the user can currently take. Accordingly, supporting the user in appropriate recovery according to their work reasonably increases the performance of daily tasks as a whole.

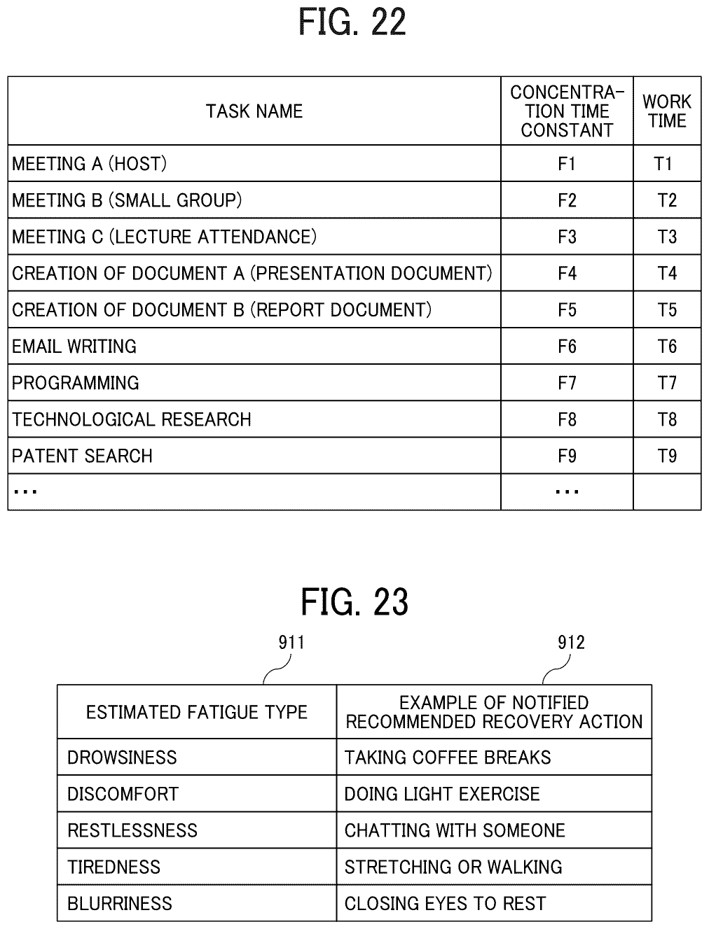

FIG. 22 is a table illustrating types of tasks and their concentration time constants. The table is obtained by calculating concentration time constants unique to the user from the history to date. In another embodiment, the table may include history information such as average work times.

FIG. 23 is a correspondence table illustrating appropriate recovery actions 912 for fatigue types 911. A recovery message screen is generated based on the table.

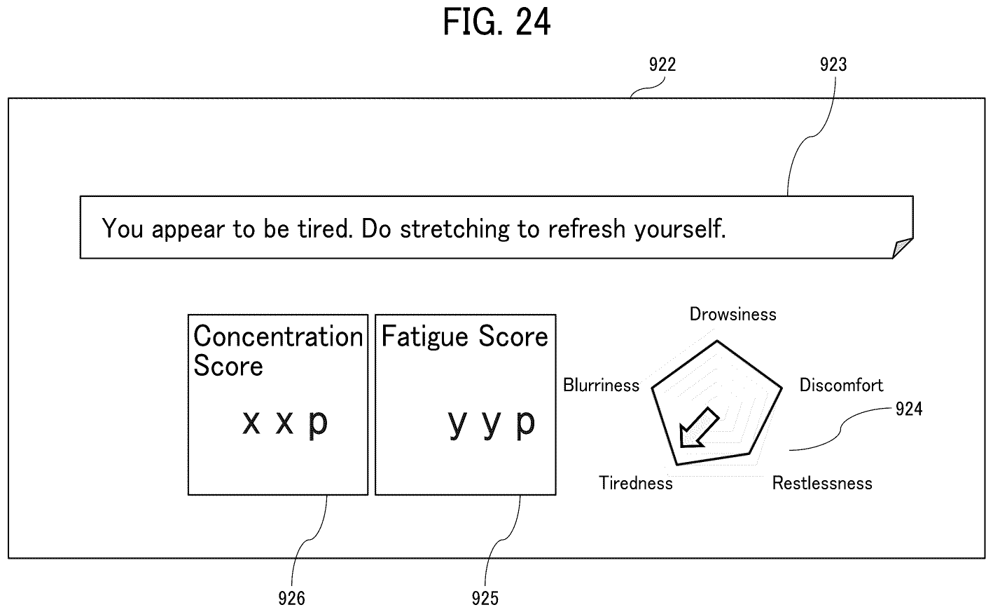

FIG. 24 illustrates an example of presenting a recommendation of an appropriate recovery action from a result obtained by the computation unit 27 (estimation unit). An example notification for prompting a user to perform a recommended recovery action in accordance with the fatigue type of the user is illustrated. For example, when the remaining energy level of the user exceeds a threshold, and the “tiredness” is estimated to be high, the computation unit 27 refers to the table illustrated in FIG. 23 and recommends the user to take a break and stretch or walk. The correspondence table between recommended actions and fatigue types illustrated in FIG. 23 is included in the recommendation information DB 2006.

In the above-described embodiments, the information analysis device 2 is the information processing device including the acquisition unit and the display control unit. However, as another embodiment, the terminal device 3 may have a function of the information processing device. In this case, the communication unit 31 of the terminal device 3 is another example of the acquisition unit. As described above, the present disclosure has following non-limiting aspects.

In Aspect 1, the information processing system 1 includes the acquisition unit 25 to acquire (step S14) information including biological information of a user, the learned model 2003 that has learned relation between biological information acquired in advance and types of mental fatigue, and the display control unit 37 to control the display 308 to display (step S30) a type of mental fatigue determined (step S18) based on the acquired information and the learned model 2003. Accordingly, a type of mental fatigue of the user is estimated and output, and the output result is satisfactory to the user. Thus, the discomfort felt by the user for the output results is reduced. The visualization of a fatigue type allows the user to take an action that prompts recovery.

In Aspect 2, the information processing system 1 includes the learned model 2003 generated, based on subjective indicators of mental fatigue or concentration acquired in advance, by learning relation between biological information and the subjective indicators of mental fatigue or concentration, the acquisition unit 25 to acquire (step S14) information including biological information of a user, and the display control unit 37 to control the display 308 to display (step S30) a subjective indicator of mental fatigue or concentration determined (step S19) (step S20) based on the acquired information and the learned model 2003. The subjective indicators correspond to the objective variables 803 illustrated in FIG. 8. The objective variables 803 are obtained by quantifying subjective indicators felt by the user, namely, “drowsiness,” “discomfort,” “restlessness,” “tiredness,” “blurriness,” “fatigue,” and “concentration.”

Accordingly, a mental fatigue indicator or a concentration level indicator, which is based on the subjectivity of the user, is estimated and output, and the output result is satisfactory to the user. Thus, the discomfort felt by the user for the output results is reduced. The visualization of the concentration level of the user allows the user to take an action of increasing the concentration level. The reduced discomfort reduces the stress felt by the user when the user uses the information processing system 1, and motivates the user to continuously use the information processing system 1.

According to Aspect 3, in the information processing system 1 of Aspect 1 or 2, the display control unit 37 determines (step S25) a remaining energy level of the user based on the subjective indicator of mental fatigue or the type of mental fatigue, and controls the display 308 to display (step S30) the remaining energy level. This configuration makes the remaining energy level visible to the user, making it easier for the user to take the next action, such as taking a break or entering a concentration mode. For example, comparing the state of a user at the start of work on each day with the state of the user at the start of daily work in the past or considering previous behaviors that the user exhibits from various aspects allows the user to easily identify the cause of a disorder experienced by the user or the cause of fatigue experienced by the user or to be easily aware of their unique tendency for disorders or fatigue. Accordingly, the user can easily find a recovery action suitable for the user, maintain health, and maintain performance. In addition, the user can optimize weekly scheduling for tasks by themselves.

According to Aspect 4, in the information processing system 1 of any one of Aspects 1 to 3, the acquisition unit 25 acquires information on schedules including tasks of a user, and the display control unit 37 controls the display 308 to display schedule information indicating a task of the user for a certain period of time and a display result of the type of mental fatigue of Aspect 1 or the subjective indicator of mental fatigue or concentration of Aspect 2 in such a manner that the display result is superimposed on the schedule information. This configuration provides simultaneous visualization of a tendency of the user and the schedules, allowing the user to select an appropriate time slot when scheduling the next task, and to efficiently perform the task. Since time slots in which the user tends to concentrate are identified in advance, the user schedules tasks while understanding the tendency. Thus, the user can perform the tasks in a well-motivated manner without being unnecessarily stressed. Fatigue levels or concentration levels associated with previous tasks are visualized to allow the user to identify a tendency, which provides an opportunity to reduce unnecessary work or meetings when, for example, sufficient time is not available.

According to Aspect 5, in the information processing system 1 of any one of Aspects 1 to 4, the display control unit 37 controls the display 308 to display a message recommending a recovery action selected based on the type of mental fatigue. This configuration makes it easier to obtain the effect of a recovery action.

According to Aspect 6, in the information processing system 1 of any one of Aspects 1 to 5, the acquisition unit 25 acquires information on a schedule including a task of the user, and the display control unit 37 controls the display 308 to display the message at a timing based on the task. This configuration allows the timing at which the user is notified of a message prompting a recovery action to be optimized in accordance with the state of the user. As a result, the user can perform a recovery action before the user unconsciously becomes too fatigued to recover.

According to Aspect 7, the information processing system 1 of any one of Aspects 1 to 6 further includes an acceleration sensor to acquire the biological information. With this configuration, the acceleration sensor, which is a contact sensor, allows sensing of respiratory data with high accuracy. In addition, the acceleration sensor is a small sensor, and the user wearing the sensor feels less stressed even when the sensor contacts the body of the user, and can continuously use the sensor.

According to Aspect 8, the information processing system 1 of any one of Aspects 1 to 7 further includes a microphone to acquire the biological information. This configuration allows respiratory data to be acquired easily at low cost. In addition, the microphone is less susceptible to body motion and is a small sensor. Thus, the user wearing the microphone feels less stressed even when the microphone contacts the body of the user, and can continuously use the microphone. A single microphone can acquire multi-modal characteristics such as a heart rate, body motion, and presence or absence of a conversation.

According to Aspect 9, in the information processing system 1 of any one of Aspects 1 to 8, it is estimated whether the user is seated. Accordingly, it is determined whether the user is seated and performing work at rest, and measurement is performed during a period of time in which it is determined that the user is seated and performing work at rest. This configuration allows a respiratory waveform signal to be accurately acquired, and improves estimation accuracy.

According to Aspect 10, in the information processing system 1 of any one of Aspects 1 to 9, it is estimated whether the user has a conversation. Accordingly, it is determined whether the user is performing work at rest without having a conversation with another person, and measurement is performed during a period of time in which it is determined that the user is performing work at rest without having a conversation with another person. This configuration allows a respiratory waveform signal to be accurately acquired, and improves estimation accuracy.

According to Aspect 11, in the information processing system 1 of any one of Aspects 1 to 10, a feature value of respiration and a feature value of a heart rate are acquired. This configuration allows multi-modal measurement and can improve the accuracy of estimating the fatigue level or the concentration level. Feature values closely related to respiration and the heart rate, such as respiratory sinus arrhythmia (RSA), can be removed.

According to Aspect 12, in the information processing system 1 of any one of Aspects 1 to 11, the learned model 2003 is augmented with information acquired by the acquisition unit 25. The acquisition unit 25 acquires information on the user. The information reflects the unique characteristics of the user. The information, which identifies the user, is further put into the learned model 2003, thereby providing invaluable information for estimating the unique characteristics of the user. Data is used in a user-specific manner to, for example, weight such additional information, thereby generating a learned model customized to a specific user. This configuration greatly improves the estimation accuracy.

In Aspect 13, an information processing method includes acquiring (step S14) information including biological information of a user; generating a learned model 2003 that has learned biological information acquired in advance and types of mental fatigue; determining (step S18) a type of mental fatigue based on the acquired information and the learned model 2003; and displaying (step S30) the determined type of mental fatigue on a display unit. Accordingly, a type of mental fatigue of the user is estimated and output, and the output result is satisfactory to the user. Thus, the discomfort felt by the user for the output results is reduced. The visualization of a fatigue type allows the user to take an action that prompts recovery.

In Aspect 14, an information processing program causes a computer to execute acquiring (step S14) information including biological information of a user; generating a learned model 2003 that has learned biological information acquired in advance and types of mental fatigue; determining (step S18) a type of mental fatigue based on the acquired information and the learned model 2003; and displaying (step S30) the determined type of mental fatigue on a display unit. Accordingly, a type of mental fatigue of the user is estimated and output, and the output result is satisfactory to the user. Thus, the discomfort felt by the user for the output results is reduced. The visualization of a fatigue type allows the user to take an action that prompts recovery. In the above Aspect 1 to 14 of the present disclosure, the display control unit 22 for displaying various kinds of information on the display 208 may be used instead of the display control unit 37 for displaying various kinds of information on the display 308.

The above-described embodiments are illustrative and do not limit the present invention. Thus, numerous additional modifications and variations are possible in light of the above teachings. For example, elements and/or features of different illustrative embodiments may be combined with each other and/or substituted for each other within the scope of the present invention. Any one of the above-described operations may be performed in various other ways, for example, in an order different from the one described above.

The functionality of the elements disclosed herein may be implemented using circuitry or processing circuitry which includes general purpose processors, special purpose processors, integrated circuits, application-specific integrated circuits (ASICs), field-programmable gate arrays (FPGAs), and/or combinations thereof which are configured or programmed, using one or more programs stored in one or more memories, to perform the disclosed functionality. Processors are considered processing circuitry or circuitry as they include transistors and other circuitry therein. In the disclosure, the circuitry, units, or means are hardware that carry out or are programmed to perform the recited functionality. The hardware may be any hardware disclosed herein which is programmed or configured to carry out the recited functionality.

There is a memory that stores a computer program which includes computer instructions. These computer instructions provide the logic and routines that enable the hardware (e.g., processing circuitry or circuitry) to perform the method disclosed herein. This computer program can be implemented in known formats as a computer-readable storage medium, a computer program product, a memory device, a record medium such as a CD-ROM or DVD, and/or the memory of an FPGA or ASIC.

1. An information processing apparatus comprising circuitry configured to:
acquire information including biological information of a user; and
cause a display to display at least one of a subjective indicator of total mental fatigue, a subjective indicator of concentration, or multiple types of mental fatigue determined based on the acquired information and a learned model,
the learned model having learned relations between biological information acquired in advance and the subjective indicator of total mental fatigue, the subjective indicator of concentration, or each of the multiple types of mental fatigue.

2. The information processing apparatus according to claim 1,
wherein the learned model has learned relations among the biological information acquired in advance, each of the multiple types of mental fatigue, and a subjective indicator of each of the multiple types of mental fatigue, and
wherein the circuitry causes the display to display the multiple types of mental fatigue and the subjective indicator of each of the multiple types of mental fatigue in association with each other.

3. The information processing apparatus according to claim 2,
wherein the circuitry causes the display to display a remaining energy level of the user determined based on the subjective indicator of total mental fatigue and the subjective indicator of each of the multiple types of mental fatigue.

4. The information processing apparatus according to claim 1,
wherein the circuitry:
acquires schedule information of the user including a task of the user; and
causes the display to display the at least one of the subjective indicator of total mental fatigue, the subjective indicator of concentration, or the multiple types of mental fatigue in a manner superimposed on the schedule information.

5. The information processing apparatus according to claim 1,
wherein the circuitry causes the display to display a message recommending taking a recovery action, the recovery action being determined based on the multiple types of mental fatigue.

6. The information processing apparatus according to claim 1,
wherein the circuitry:
acquires schedule information of the user including a task of the user; and
causes the display to display a message recommending taking a recovery action at a timing based on the task.

7. An information processing method comprising:
acquiring information including biological information of a user; and
causing a display to display at least one of a subjective indicator of total mental fatigue, a subjective indicator of concentration, or multiple types of mental fatigue determined based on the acquired information and a learned model,
the learned model having learned relations between biological information acquired in advance and the subjective indicator of total mental fatigue, the subjective indicator of acquiring information including biological information of a user; and concentration, or each of the multiple types of mental fatigue.

8. A non-transitory recording medium storing a plurality of instructions which, when executed by one or more processors, causes the processors to perform a method, the method comprising:
acquiring information including biological information of a user; and
causing a display to display at least one of a subjective indicator of total mental fatigue, a subjective indicator of concentration, or multiple types of mental fatigue determined based on the acquired information and a learned model,
the learned model having learned relations between biological information acquired in advance and the subjective indicator of total mental fatigue, the subjective indicator of concentration, or each of the multiple types of mental fatigue.
